(19) United States
(12) Patent Application Publication
(10) Pub. No.: US 2022/0105174 A1
(43) Pub. Date: Apr. 7, 2022

(54) RECOMBINANT GALLID HERPESVIRUS 3 VACCINES ENCODING HETEROLOGOUS AVIAN PATHOGEN ANTIGENS

(71) Applicant: The Pirbright Institute, Pirbright (GB)

(72) Inventors: Yashar Sadigh, Pirbright (GB); Venugopal Nair, Pirbright (GB)

(73) Assignee: The Pirbright Institute, Pirbright (GB)

(21) Appl. No.: 17/493,643

(22) Filed: Oct. 4, 2021

Related U.S. Application Data
(63) Continuation of application No. 16/606,502, filed on Oct. 18, 2019.

(30) Foreign Application Priority Data
Apr. 21, 2017 (EP) 17167638.0

(51) Int. Cl. A61K 39/255; A61K 39/17; A61K 39/295; C12N 15/86

(52) U.S. Cl. CPC A61K 39/255; A61K 39/17; A61K 2039/5256; C12N 15/86; A61K 39/295

(57) ABSTRACT

The invention relates to a recombinant Gallid herpesvirus 3 vector encoding heterologous avian pathogen antigens comprising one or more heterologous polynucleotide(s) inserted into the intergenic loci UL3/UL4 and/or UL21/UL22. The invention further relates to vaccines comprising said recombinant Gallid herpesvirus 3 vector and optionally a further Marek's disease virus vector and to a use of the vaccine for protecting an avian species against one or more avian pathogens. Further methods for treating an avian species for protection against one or more diseases caused by avian pathogens and a method for producing the recombinant Gallid herpesvirus 3 vector encoding heterologous avian pathogen antigens is provided.

### CROSS-REFERENCE TO RELATED APPLICATIONS

This application is a continuation of U.S. application Ser. No. 16/606,502, filed Oct. 18, 2019, which is a national phase entry pursuant to 35 U.S.C. § 371 of International Application No. PCT/EP2018/060225, filed Apr. 20, 2018, which claims priority from European Application No. EP 17167638.0, filed Apr. 21, 2017, the entire contents of which are incorporated by reference herein for all purposes.

The instant application contains a Sequence Listing which has been submitted electronically in ASCII format and is hereby incorporated by reference in its entirety. Said ASCII copy, created Nov. 12, 2021, is named 01251-0003-01US_SequenceListing and is 70 bytes in size.

### TECHNICAL FIELD

The invention relates to a recombinant Gallid herpesvirus 3 vector encoding heterologous avian pathogen antigens comprising one or more heterologous polynucleotide(s) inserted into the intergenic loci UL3/UL4 and/or UL21/UL22. The invention further relates to vaccines comprising said recombinant Gallid herpesvirus 3 vector and optionally a further Marek's disease virus vector and to a use of the vaccine for protecting an avian species against one or more avian pathogens. Further methods for treating an avian species for protection against one or more diseases caused by avian pathogens and a method for producing the recombinant Gallid herpesvirus 3 vector encoding heterologous avian pathogen antigens is provided.

### BACKGROUND

Marek's disease (MD) is a highly contagious disease of poultry characterised by rapid-onset of T-cell lymphomas. The disease has a worldwide distribution and causes huge economic losses to the poultry industry. The causative agent of the disease, Marek's disease virus-1 (MDV-1, Gallid herpesvirus 2, GaHV-2), is an alpha herpesvirus of the genus Mardivirus (ICTV, 2006), which also includes the antigenically related herpesvirus of turkeys (HVT, Meleagrid herpesvirus 1), a strain used widely as a vaccine against MD since the late 1960s (Kawamura et al., 1969; Witter et al., 1970). The third member of the genus Mardivirus is the MDV-2 (Gallid herpesvirus 3, GaHV-3), which includes apathogenic strains some of which are used as live vaccines against MD (von Billow et al., 1975).

Marek's disease virus-1 (MDV-1) or Gallid herpesvirus 2 (GaHV2), the causative agent of Marek's disease (MD), is one of the major pathogens in poultry. MDV-1 is a member of the Mardivirus genus in the family Herpesviridae. In the infected birds, MDV-1 replicates in the feather follicle epithelial cells and spreads through the respiratory route by the inhalation of the poultry house dust contaminated with infectious virus shed with the dead skin and dander. MD is characterized by widespread T cell lymphomas and paralytic symptoms due to neuronal infiltration of lymphocytes. The mortality rate due to MD usually varies between 10-30%, but can go up to 60-80%. Live vaccines comprising of different strains are used in different combinations for the control of MD in the last 40 years (Witter R L. Curr Top Microbiol Immunol. 2001; 255: 57-90; Calnek B W et al., Avian diseases. 1983; 27: 844-9). These include the naturally attenuated MDV-1 strain Rispens (CVI-988), MDV-2 (GaHV3) strain SB-1 and herpesvirus of turkeys (HVT) strain Fc126.

In addition to their use as successful vaccines inducing long-term protection against MD, avian herpesvirus vaccine strains have also been recognized as recombinant viral vectors for inducing protection against other major avian infectious diseases. The most successful and widely used recombinant vaccine vector is HVT, which has been shown to be very effective in protecting against a number of avian viral pathogens (Morgan R W, et al., Avian diseases 1993; 37: 1032-40; Li Y, et al., Vaccine. 2011; 29: 8257-66; Darteil Ret al., Virology 1995;211:481-90; Kapczynski D R et al., Vaccine. 2015; 33: 1197-205). Although individual recombinant HVT vaccines have proven to be extremely effective, when using more than one HVT-vector vaccine it has been problematic to get the desired immune responses against each component of the combined vaccine. There are clear recommendations not to use other HVT with the recombinant vectors, as there will be interference resulting in poor efficacy against the foreign insert (American Association of Avian Pathologists A. Frequently asked questions on viral tumor diseases http://www.aaap.info/frequently-asked-questions-on-viral-tumor-diseases, 2012). With this constraint on the HVT vector for its use as multivalent vaccines, there is a need for other vector platforms that will complement rather than interfere with protection against multiple components in the vaccine.

The first GaHV3 (MDV-2) that was licensed for use as a vaccine was the SB-1 strain (Schat K A et al., J Natl Cancer Inst. 1978; 60: 1075-82). It was originally introduced as a vaccine against MD in the mid-1980s and used in combination with HVT vaccine (Calnek B W et al., Avian diseases. 1983; 27: 844-9). Bivalent vaccines containing SB-1 and HVT Fc126 strains have been successfully used in many countries including USA, South America and Asia (Witter R L. Curr Top Microbiol Immunol. 2001; 255: 57-90; Bublot M, Sharma J. Vaccination against Marek's disease. In: Davison F, Nair V, editors. Marek's Disease-An Evolving Problem. London: Elsevier Academic Press; 2004. p. 168-85). SB-1/HVT bivalent vaccines are thought to provide superior protection through a synergistic effect although the molecular mechanism has not been identified (Calnek B W et al., Avian diseases. 1983; 27: 844-9; Witter R L et al., Avian pathology: journal of the WVPA. 1984; 13: 75-92). SB-1 has also been reported to induce very good protection even in maternal antibody-positive chicks (Witter R L et al., Avian pathology: journal of the WVPA. 1984; 13: 75-92). The 166-Kb genome of the SB-1 strain of GaHV3 has a similar genome organization as MDV-1 sharing a number of homologous genes (Spatz S J et al., Virus Genes. 2011; 42: 331-8) as well as unique set of genes and microRNAs (Yao Yet al., J Virol. 2007; 81: 7164-70).

Unlike MDV-1, where extensive studies have been carried out to examine the molecular determinants of various biological characteristics, only limited studies have been carried out on the GaHV3 (MDV-2) genome to unravel some of the unique properties associated with this virus. Determination of the complete genome sequence of the MDV-2 prototype strain HPRS-24 has enabled the comparisons of the genome structure and sequence with other MDV species (Izumiya et al., 2001). Recent advances in cloning of herpesvirus genomes as bacterial artificial chromosomes (BAC) have helped to identify molecular characteristics of several herpesviruses (Adler et al., 2003; Zelnik, 2003) including MDV-1 (Petherbridge et al., 2004, 2003; Schumacher et al., 2000), HVT (Baigent et al., 2006) and MDV-2 vaccine strain SB-1 (Petherbridge et al., 2009).

Vaccines against MDV based on a recombinant herpesvirus MDV strain offers the advantage of simultaneously achieve immunity against MDV and at least a further viral pathogen. Different methods have been employed to produce such bivalent vaccines. One of the widely used and successful approaches has been the delivery of the IBDV VP2 antigen in various MDV vaccine vector platforms, including the HVT (Tsukamoto K et al., J Virol. 2002; 76: 5637-45; Perozo F et al., Avian diseases. 2009; 53: 624-8), MDV-1 (Tsukamoto K et al., Virology. 1999; 257: 352-62; Zhou X et al., Vaccine. 2010; 28: 3990-6) and fowl pox viral vectors (Tsukamoto K et al., Virology. 2000; 269: 257-67). Considering the need for protecting poultry against multiple pathogens, there is a need for additional vector platforms that will not interfere with each other to deliver protective immune responses to all the components in the vaccine.

MDV and IBDV are highly infectious viruses whose high mortality rates have made them a constant threat to the worldwide poultry industry for decades. Presence of maternal antibodies, emergence of new variants, cost of production and in some cases lack of compliance with DIVA strategy (reviewed in Muller H, et al., Avian pathology: journal of the WVPA. 2012; 41: 133-9) are challenges that limit the efficient control of IBDV. IBDV is one of the most difficult viruses to protect against using live recombinant avian vaccines and hence suitable protecting efficacy needs to be shown. Bivalent MDV/IBDV vaccines allow for vaccination against both diseases simultaneously, lowering the costs of production and inoculation. They are also safer and can be given in ovo, unlike attenuated IBDV vaccines that cause subclinical IBD in chicks and are fatal to embryos. VAXXITEK_{HVT+}IBD is an existing MDV/IBDV bivalent vaccine based on HVT. However there is still a need for further bivalent MDV vaccines.

### SUMMARY OF THE INVENTION

Provided herein is a recombinant Gallid herpesvirus 3 (GaHV3; MDV-2) vector comprising one or more heterologous polynucleotide(s) coding for and expressing at least one antigen of an avian pathogen, inserted into the intergenic loci UL3/UL4 and/or UL21/UL22 of the Gallid herpesvirus 3 vector. Preferably the recombinant Gallid herpesvirus 3 vector is a recombinant Gallid herpesvirus 3 strain SB-1 vector. In one embodiment the recombinant Gallid herpesvirus 3 vector comprises the one or more heterologous polynucleotide(s) inserted into the intergenic locus UL3/UL4 of the Gallid herpesvirus 3 vector. Preferably the at least one antigen is protective against infectious bursal disease virus (IBDV), infectious laryngotracheitis virus (ILTV), Newcastle disease virus (NDV), avian influenza virus (AIV) or avian infectious bronchitis virus (IBV). The at least one antigen may be selected from the group consisting of (a) VP2, VP3, VP4 and VPX of infectious bursal disease virus (IBDV); (b) glycoprotein B, glycoprotein I, glycoprotein D, glycoprotein E and glycoprotein C of ILTV; (c) Newcastle disease virus fusion protein (NDV-F) and viral hemagglutinin neuraminidase (NDV-NH) of NDV (d) Avian influenza hemagglutinin (HA) and neuraminidase (NA); and (e) 51 or S2 protein of IBV. Preferably the at least one antigen is protective against IBDV. More preferably the at least one antigen is VP2 of IBDV. In one embodiment the VP2 protein amino acid sequence has at least 80% sequence identity to the sequence set forth in SEQ ID

NOs: 12. In another embodiment the VP2 protein has the sequence set forth in SEQ ID NOs: 12.

The Gallid herpesvirus 3 vector preferably contains an expression cassette(s) containing the one or more heterologous polynucleotide(s), wherein optionally the expression cassette further comprises a promoter. In one embodiment the promoter is selected from the group consisting of immediate early cytomegalovirus (CMV) promoter, guinea pig CMV promoter, murine CMV promoter, SV40 promoter, pseudorabies virus promoters of glycoprotein X promoter, herpes simplex virus-1 alpha 4 promoter, chicken beta-actin promoter, rabbit beta-globin promoter, herpes simplex virus thymidine kinase promoter, Marek's Disease Virus promoters of glycoproteins gC, gB, gE, or gI genes and infectious laryngotracheitis virus promoters of glycoprotein gB, gE, gI, gD genes.

In another aspect a vaccine is provided comprising the recombinant Gallid herpesvirus 3 vector of the invention. The vaccine may further comprise a pharmaceutically acceptable excipient, carrier or adjuvant. In one embodiment the vaccine also comprises a further Marek's disease virus (MDV) vector selected from the group consisting of Gallid herpesvirus 3 vector, naturally attenuated MDV-1 strain Rispens (CVI-988) vector and herpesvirus of turkeys (HVT) strain Fc126 vector, wherein the further MDV vector may be a recombinant MDV vector. In a specific embodiment the further Marek's disease virus

(MDV) vector is a recombinant Marek's disease virus (MDV) vector comprising one or more heterologous polynucleotide(s) coding for and expressing at least one antigen of an avian pathogen. According to the invention the further recombinant Marek's disease virus vector may be a second recombinant Gallid herpesvirus 3 vector in addition to the first recombinant Gallid herpesvirus 3 vector according to the invention, preferably the second recombinant Gallid herpesvirus 3 vector is a recombinant Gallid herpesvirus 3 strain SB-1 vector.

In one embodiment the second recombinant Gallid herpesvirus 3 vector also comprises the one or more heterologous polynucleotide(s) inserted into the intergenic loci UL3/UL4 and/or UL21/UL22 of said second Gallid herpesvirus 3 vector, preferably into the intergenic locus UL3/UL4 of the second Gallid herpesvirus 3, wherein the second recombinant Gallid herpesvirus 3 vector comprises at least one heterologous polynucleotide coding for and expressing a different antigen of an avian pathogen as the one or more heterologous polynucleotides of the first recombinant Gallid herpesvirus 3 vector. The further Marek's disease virus vector and the (first) recombinant Gallid herpesvirus 3 vector according to the invention may be administered together or separate from each other.

In another aspect the present invention provides an isolated DNA encoding the recombinant Gallid herpesvirus 3 vector according to the invention

In yet another aspect the present invention provides a bacterial artificial chromosome (BAC) comprising a polynucleotide coding for the recombinant Gallid herpesvirus 3 vector according to the invention.

In yet another aspect the vaccine of the invention is provided for use in vaccinating an avian species against one or more diseases caused by one or more avian pathogens, preferably against Marek's disease and one or more diseases caused by one or more avian pathogens. In one embodiment the one or more diseases are caused by one or more of infectious bursal disease virus (IBDV), infectious laryngotracheitis virus (ILTV), Newcastle disease virus (NDV), avian influenza virus (AIV) or avian infectious bronchitis virus (IBV). The vaccine is further provided for use in protecting an avian species against clinical symptoms caused by one or more avian pathogens, preferably against clinical symptoms caused by Marek's disease virus and clinical symptoms caused by one or more avian pathogens. In one embodiment the one or more avian pathogen causing the diseases or clinical symptoms is selected from the group consisting of Newcastle disease virus, infectious bursal disease virus and avian infectious laryngotracheitis virus, avian influenza virus and avian infectious bronchitis virus.

The avian species may be poultry, preferably the avian species is chicken, duck, goose, turkey, quail, guinea or pigeon, more preferably the avian species is turkey or chicken, even more preferably chicken. The vaccine according to the invention may be administered by spray administration, in ovo, subcutaneously, intramuscularly, orally or nasally. In a particular embodiment the vaccine is administered in ovo preferably in ovo in 18 day old embryonated eggs. In an alternative embodiment the vaccine is administered in subcutaneously or intramuscularly in chicks, preferably in 1 day old chicks.

Further provided is a method of treating an avian species for protection against Marek's Disease and one or more diseases caused by one or more avian pathogens comprising the step of administering an effective amount of the vaccine according to the invention, wherein preferably the one or more diseases is caused by one or more of infectious bursal disease virus (IBDV), infectious laryngotracheitis virus (ILTV), Newcastle disease virus (NDV), avian influenza virus (AIV) or avian infectious bronchitis virus (IBV).

In yet another aspect a recombinant Gallid herpesvirus 3 (GaHV3; MDV-2) vector is provided comprising one or more marker(s) inserted into the intergenic loci UL3/UL4 and/or UL21/UL22 of the Gallid herpesvirus 3 vector, preferably into the intergenic locus UL3/UL4 of the Gallid herpesvirus 3 vector, wherein the marker is preferably a selection marker gene, a reporter gene or a DNA bar code. Preferably, the recombinant Gallid herpesvirus 3 vector is a recombinant Gallid herpesvirus 3 strain SB-1 vector. Also provided is a bacterial artificial chromosome (BAC) comprising a polynucleotide coding for the recombinant Gallid herpesvirus 3 vector comprising one or more marker(s) inserted into the intergenic loci UL3/UL4 and/or UL21/UL22 of the Gallid herpesvirus 3 vector, wherein the recombinant Gallid herpesvirus 3 vector is preferably a recombinant Gallid herpesvirus 3 strain SB-1 vector.

Also provided is a method of producing a recombinant Gallid herpesvirus 3 vector comprising (a) providing a Gallid herpesvirus 3 vector, (b) inserting one or more heterologous polynucleotide(s) coding for and expressing at least one antigen of an avian pathogen into the intergenic loci UL3/UL4 and/or UL21/UL22 of the Gallid herpesvirus 3 vector, and optionally (c) amplifying the Gallid herpesvirus 3 vector comprising one or more heterologous polynucleotide(s) coding for at least one antigen of an avian pathogen of step (b). In one embodiment the method comprises (a) providing a Gallid herpesvirus 3 vector comprising one or more marker(s) in the intergenic loci UL3/UL4 and/or UL21/UL22 of the Gallid herpesvirus 3 vector, (b) replacing the one or more marker(s) with an expression cassette comprising one or more heterologous polynucleotides coding for at least one antigen of an avian pathogen, and (c) amplifying the Gallid herpesvirus 3 vector comprising one or more heterologous polynucleotide(s) coding for at least one antigen of an avian pathogen of step (b). Preferably, the recombinant Gallid herpesvirus 3 vector is a recombinant Gallid herpesvirus 3 strain SB-1 vector.

### FIGURE LEGENDS

FIG. 1. A) Recombineering process to generate (top, A-i) SB-1-UL3/4VP2, (middle, A-ii) SB-1-UL10/11VP2 and (bottom, A-iii) SB-1-UL21/22VP2. The VP2 expression cassette was inserted in the intergenic loci UL3/UL4, UL10/UL11 and UL21/UL22. The VP2 expression cassette is shown as a solid arrow. The location and the orientation of the genes from SB-1 is shown. B) VP2 immunostaining (dark blue) in plaques of infected cells. CEF cells were infected with (B-i) SB-1-UL3/4VP2, SB-1-UL21/22VP2 and (B-iii) SB-1-UL10/11VP2. Cells were fixed five days post infection and stained as described in the materials and methods section (scale bar=2000 μm).

FIG. 2. Growth curve for SB-1 recombinant viruses in vitro. The genome copy number per one million CEF cells is plotted against hours post infection; SB-1-UL3/4VP2 (▪), SB-1-UL21/22VP2 (●), SB-1-UL10/11VP2 (◯) and SB-1 (▴) vaccine viruses. No significant difference between SB-1 and SB-1 UL3/4VP2 after 24 hours post infection was observed. Replication level for SB-1-UL10/11VP2 and SB-1-UL21/22VP2 were lower than that of SB-1 and SB-1-UL3/4VP2 viruses. Each time point was calculated based on three replicates. Each replicate was measured in triplicate. Error bars represent the standard deviation.

FIG. 3. Titers of neutralizing antibodies against VP2 in the sera of chickens vaccinated with VAXXITEK_{HVT+IBD} (♦), SB-1-UL3/4VP2 (▪), SB-1-UL21/22VP2 (●), SB-1-UL10/11VP2 (◯) and SB-1 (▴) vaccine viruses. Serum samples were collected at weeks 2, 3 and 4 post-vaccination. The mean titer in each group is shown as a horizontal bar.

FIG. 4. Chickens were vaccinated with VAXXITEK_{HVT-HBD} (♦), SB-1-UL3/4VP2 (▪), SB-1-UL21/22VP2 (●) and SB-1 (▴) followed by infection with IBDV UK661 at time point 0. A) Mean clinical score in vaccinated birds challenged with IBDV UK661. Bars show standard deviation, n=8. B) Percentage survival for the vaccinated birds challenged with IBDV UK661. Birds inoculated with pSB-1 were euthanized for humane reasons or died 55 hours post IBDV infection.

FIG. 5. Chickens were vaccinated with SB-1-UL3/4VP2 (▪), SB-1-UL3/4VP2 and HVT-9HA (▴) and no vaccine (negative control) (●). Titers of neutralizing antibodies against IBDV VP2 at weeks 0, 1, 3, 4, 5 and 6 are shown (n=10). Error bars represent the standard deviation.

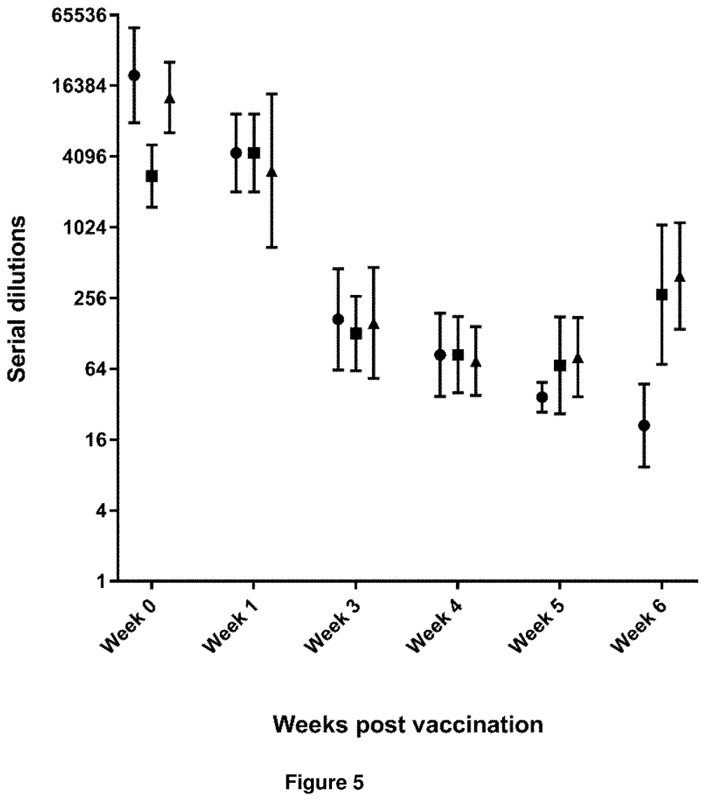

### DETAILED DESCRIPTION

The general embodiments “comprising” or “comprised” encompass the more specific embodiment “consisting of”. Furthermore, singular and plural forms are not used in a limiting way. As used herein, the singular forms “a”, “an” and “the” designate both the singular and the plural, unless expressly stated to designate the singular only.

The term “animal” is used herein to include all mammals, birds and fish. In this particular context animal refers to an avian species, such as chicken, duck, goose, turkey, quail, guinea, pigeon, swan, pheasant, parrot, finch, hawk, crow, ostrich, emu and cassowary. The term “animal” and “avian” also includes an individual animal in all stages of development, including embryonic and fetal stages. Preferably, the animal is poultry. As used herein the term “poultry” refers to a domestic or commercial bird kept for the eggs they produce, their meat or feathers. Poultry may be birds from the order Galliformes, such as chicken, duck, goose, turkey, quail, guinea and pigeon.

The term “avian species” as used herein relates to birds, preferably poultry, such as chicken, duck, goose, turkey, quail, guinea or pigeon. Particularly preferred in the context of the present invention is turkey or chicken, even more preferred chicken.

The terms “nucleic acid”, “nucleotide”, and “polynucleotide” as used herein are used interchangeably and refer to a single or double- stranded polymer of deoxyribonucleotide bases or ribonucleotide bases read from the 5′ to the 3′ end and include RNA, DNA, cDNA, or cRNA and derivatives thereof, such as those containing modified backbones. In the context of the Gallid herpesvirus 3 vector the skilled person would understand that it refers to deoxyribonucleic acid, i.e., DNA or cDNA.

Polynucleotides according to the invention can be prepared in different ways (e.g. by chemical synthesis, by gene cloning etc.) and can take various forms (e.g. linear or branched, single or double stranded, or a hybrid thereof, primers, probes etc.). The term “nucleotide sequence” or “nucleic acid sequence” refers to both the sense and antisense strands of a nucleic acid as either individual single strands or in the duplex. The term “ribonucleic acid” (RNA) is inclusive of RNAi (inhibitory RNA), dsRNA (double stranded RNA), siRNA (small interfering RNA), mRNA (messenger RNA), miRNA (micro-RNA), tRNA (transfer RNA, whether charged or discharged with a corresponding acylated amino acid), and cRNA (complementary RNA).

The term “genomic DNA”, or “genome” is used interchangeably and refers to the heritable genetic information of a host organism. The genomic DNA comprises the DNA of the nucleus (also referred to as chromosomal DNA) but also of other cellular organelles (e.g., mitochondria).

The term “genomic RNA” or “genome” refers to the heritable genetic information of an RNA virus. The genomic RNA may be a positive strand or a negative strand RNA.

The term “gene” as used herein refers to a DNA or RNA locus of heritable genomic sequence which affects an organism's traits by being expressed as a functional product or by regulation of gene expression. Genes and polynucleotides may include introns and exons as in genomic sequence, or just the coding sequences as in cDNAs, such as an open reading frame (ORF), comprising a start codon (methionine codon) and a translation stop codon. Genes and polynucleotides can also include regions that regulate their expression, such as transcription initiation, translation and transcription termination. Thus, also included are regulatory elements such as promoters.

The term “heterologous polynucleotide” as used herein refers to a polynucleotide derived from a different organism or a different species from the recipient coding for a heterologous protein. In the context of the Gallid herpesvirus 3 vector the skilled person would understand that it refers to a DNA or cDNA. A heterologous polynucleotide may also be referred to as transgene. Thus, it may be a gene or open reading frame (ORF) coding for a heterologous protein. In the context of the Gallid herpesvirus 3 “heterologous polynucleotide” refers to a polynucleotide derived from a different avian pathogen or virus (different species and/or strain), particularly a different avian virus, including a different virus of the family Herpesviridae that causes avian infection and a different strain of Gallid herpesvirus 3. The term “heterologous” when used with reference to portions of a nucleic acid indicates that the nucleic acid comprises two or more sequences that are not found in the same relationship to each other in nature.

Heterologous may also refer to a viral polynucleotide sequence, such as a gene or transgene, or a portion thereof, being inserted into a viral genome in which it is not typically found, or a gene introduced into an organism in which it is not typically found.

A “recombinant viral vector” or “viral vector” as used herein refers to a recombinant virus comprising a virus genome and a heterologous polynucleotide for transduction of a host cell. Such a recombinant viral vector according to the invention may be derived from a Gallid herpesvirus 3 strain SB-1 or HPRS24. As appropriate, viral gene- or protein-coding sequences may be incorporated into such a recombinant viral vector as described herein for vaccinating a chicken or other poultry against one or more viral diseases. A recombinant viral vector may also comprise a heterologous polynucleotide coding for and expressing a marker. Typically a recombinant viral vector contains a heterologous polynucleotide or transgene that is operatively linked to a promoter in order to effect transcription of the transgene. The term “recombinant” further includes any modification, alteration or engineering of a polynucleotide or protein in its native form or structure, or any modification, alteration or engineering of a polynucleotide or protein in its native environment or surrounding. The modification, alteration or engineering of a polynucleotide or protein may include, but is not limited to, deletion of one or more nucleotides or amino acids, deletion of an entire gene, codon-optimization of a gene, conservative substitution of amino acids and insertion of one or more heterologous polynucleotides.

The term “recombinant Gallid herpesvirus 3 (GaHV3, MDV-2) vector” as used herein refers to a recombinant virus comprising all essential genes of GaHV3, such as of strain SB-1 or HPRS24. The Gallid herpesvirus 3 (MDV-2) strains used for the recombinant viral vector may be any SB-1 strains, including, but not limited to, the commercial Marek's Disease Vaccine (SB-1 vaccine) (Merial Select Inc., Gainesville, Ga. 30503, USA), having a genome sequence as defined by GenBank Accession Number HQ840738.1 The recombinant Gallid herpesvirus 3 (GaHV3, MDV-2) vector may further contain deleted or mutated non-essential genes. The Gallid herpesvirus 3 (MDV-2) may be any Gallid herpesvirus 3 (MDV-2) comprising a genome sequence having at least 95%, 96%, 97%, 98%, or 99% sequence identity to the sequence of Gallid herpesvirus 3 (MDV-2) strain SB-1 as defined in GenBank Accession Number HQ840738.1 (without considering inserted heterologous polynucleotide sequences). Also deletion of any non-essential genes is not considered for sequence alignment.

A Gallid herpesvirus 3 (MDV-2) strain other than SB-1 is the HPRS24 strain having the genome sequence as defined by GenBank Accession Number NC_002577.1 (INSDC: AB049735.1). The genomes of HPRS24 and SB-1 share 98.4% sequence identity (Spatz and Schat, 201 1; Virus Gene 42, 331-338). The Gallid herpesvirus 3 (MDV-2) strains used for the recombinant viral vector may be the HN-1, 287C/1, 401/1, 437A/1, 301B/1, 471B/1, 281MI/1 and 2986/1 isolates described in the publications (Witter (1987) Avian Dis 31, 752-765; Witter et al. (1987) Avian Dis 31, 829-840; Witter (1995) Avian Pathology (1995) 24, 665-678). Although not used as a vaccine strain to date, strain HPRS24, or any of the above mentioned MDV-2 isolates can also considered a suitable vaccine strain, due to their relatedness and antigenic similarity.

The term “protein” is used interchangeably with “amino acid residue sequences” or “polypeptide” and refers to polymers of amino acids of any length. These terms also include proteins that are post-translationally modified through reactions that include, but are not limited to, glycosylation, acetylation, phosphorylation or protein processing. Modifications and changes, for example fusions to other proteins, amino acid sequence substitutions, deletions or insertions, can be made in the structure of a polypeptide while the molecule maintains its biological functional activity. For example certain amino acid sequence substitutions can be made in a polypeptide or its underlying nucleic acid coding sequence and a protein can be obtained with the same properties.

The term “antigen” as uses herein is a substance which provokes an adaptive immune response in a host animal and may also be referred to as immunogen. Antigens are typically of high molecular weight and proteins or polysaccharides. Peptides, lipids, nucleic acids and many other materials can also function as antigens. An antigen can be a whole organism, killed, attenuated or live or a subunit or portion of an organism; a piece or fragment of DNA capable of inducing an immune response upon presentation to a host animal; a polypeptide or a fragment thereof, an epitope, a hapten, or any combination thereof. An immunogen or antigen may be also a toxin or antitoxin. The antigen coded for and expressed by the heterologous polynucleotides according to the invention is typically an immunogenic or antigenic protein. As used herein an antigen may also refer to the immunogenic part or fragment of an antigen comprising the epitope, e.g., peptide(s).

As used herein the term “antigen of an avian pathogen” refers to a protein encoded by a pathogen, preferably a virus described herein, including structural and non-structural proteins. Such antigens may include naturally occurring or non-naturally occurring viral proteins from IBDV, NDV, AIV, ILTV and/or IBV including VP2, F, and/or HN proteins. As used herein, an “antigen” refers to a viral protein or polypeptide, such as a viral polypeptide, as well as viral particles. In some embodiments, an antigen in accordance with the invention may also be a viral nucleic acid.

The term “immunogenic” protein or peptide as used herein includes polypeptides that are immunologically active in the sense that once administered to the host, it is able to evoke an immune response of the humoral and/or cellular type directed against the protein. Preferably the protein fragment is such that it has substantially the same immunological activity as the full length protein. Thus, a protein fragment according to the invention comprises or consists essentially of or consists of at least one epitope or antigenic determinant. An “immunogenic” protein or polypeptide, as used herein, includes the full-length sequence of the protein, analogs thereof, or immunogenic fragments thereof. By “immunogenic fragment” is meant a fragment of a protein which includes one or more epitopes and thus elicits the immunological response described above.

The term “immunogenic protein or peptide” further contemplates deletions, additions and substitutions to the sequence, as long as the polypeptide functions to produce an immunological response as defined herein. The term “conservative variation” denotes the replacement of an amino acid residue by another biologically similar residue, or the replacement of a nucleotide in a nucleic acid sequence such that the encoded amino acid residue does not change or is another biologically similar residue. In this regard, particularly preferred substitutions will generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine, threonine, tyrosine. Phenylalanine, tryptophan, and tyrosine are sometimes classified as aromatic amino acids. Examples of conservative variations include the substitution of one hydrophobic residue such as isoleucine, valine, leucine or methionine for another hydrophobic residue, or the substitution of one polar residue for another polar residue, such as the substitution of arginine for lysine, glutamic acid for aspartic acid, or glutamine for asparagine, and the like; or a similar conservative replacement of an amino acid with a structurally related amino acid that will not have a major effect on the biological activity. Proteins having substantially the same amino acid sequence as the reference molecule but possessing minor amino acid substitutions that do not substantially affect the immunogenicity of the protein are, therefore, within the definition of the reference polypeptide. All of the polypeptides produced by these modifications are included herein. The term “conservative variation” also includes the use of a substituted amino acid in place of an unsubstituted parent amino acid provided that antibodies raised to the substituted polypeptide also immunoreact with the unsubstituted polypeptide.

The term “epitope” refers to the site on an antigen or hapten to which specific B cells and/or T cells respond. The term is also used interchangeably with “antigenic determinant” or “antigenic determinant site”. Antibodies that recognize the same epitope can be identified in a simple immunoassay showing the ability of one antibody to block the binding of another antibody to a target antigen. An epitope may be linear or conformational. A conformational epitope is composed of discontinuous sections of the antigen's amino acid sequence.

An “immunological response” to a composition or vaccine is the cellular and/or antibody-mediated immune response elicited by and to a composition or vaccine of interest. Usually, an “immunological response” includes but is not limited to one or more of the following effects: the production of antibodies, B cells, helper T cells, and/or cytotoxic T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

The term “avian pathogen” as used herein refers to an infectious agent that is pathogenic in birds, including bacteria, viruses, yeast, nematodes, fungi etc. Particularly preferred in the present context are avian viruses and avian bacteria and more preferred avian viruses. Avian viruses of particular interest are infectious bursal disease virus (IBDV), New castle disease virus (NDV), infectious laryngotracheitis virus (ILTV), avian influenza virus (AIV) and avian infectious bronchitis virus (IBV).

As used herein, an “insertion site” refers to a region in a viral genome into which a heterologous polynucleotide is inserted and which is a nonessential region. The insertion sites of the present invention may be an intergenic region or intergenic locus, particularly the intergenic locus between UL3 and UL4 and/or the intergenic locus between UL21 and UL22 in the unique long (UL) region of the genome, preferably the intergenic locus between UL3 and UL4. Insertion of one or more heterologous polynucleotides into one of these regions enables the production of a recombinant viral vector that can then be introduced into a chicken or other poultry for protection against one or more diseases. In some embodiments, a heterologous polynucleotide coding for and expressing an antigen of an avian pathogen as described herein may be inserted at an insertion site as disclosed herein in addition to one or more insertion sites known in the art. A suitable insertion site should allow insertion of a polynucleotide without affecting replication and growth of the virus. The skilled person will understand that in the context of a vaccine a suitable insertion site should also allow that the inserted polynucleotide elicits an appropriate immune response and protection against the respective avian pathogen.

The term “nonessential region” refers to a region of a virus genome which is not essential for replication and propagation of the virus in tissue culture of the host. Theoretically, any nonessential region or portion thereof can be deleted from the Gallid herpesvirus 3 strain SB-1 or HPRS24 genome or a foreign sequence can be inserted in it, and the viability and stability of the recombinant Gallid herpesvirus 3 vector resulting from the deletion or insertion can be used to ascertain whether a deleted region or portion thereof is indeed nonessential.

The term “intergenic locus” as used herein refers to the region between two genes and in the context of the present invention to the locus between two genes of the unique long (UL) region of a Gallid herpesvirus 3 (GaHV3, MDV-2) vector. Insertion into the intergenic locus at a specific location means that the transgene or heterologous polynucleotide is inserted between two genes, more specifically between two genes of the UL region, without replacing or deleting a gene. For example, the intergenic locus of UL3/UL4 and UL21/UL22 has a sequence as provided in SEQ ID NO: 1 and 2, respectively.

As used herein, the term “expression cassette” refers to the part of a vector comprising all elements required for the expression of a polynucleotide in a host cell. It contains one or more gene(s) coding for a protein and sequences controlling the expression, i.e. regulatory elements. Thus it comprises a promoter operably linked to an open reading frame (ORF) and a 3′ untranslated region containing a transcription termination region. Additional elements are, e.g., an enhancer. The expression cassette may be part of a vector, typically an expression vector, including a plasmid or a viral vector. It may also be integrated in a chromosome by random or targeted integration, such as by homologous recombination or by viral integration. An expression cassette is prepared using cloning techniques and does therefore not refer to a natural occurring gene structure.

As used herein, the term “promoter” refers to a region of DNA that initiates transcription of a particular gene or open reading frame. Promoters are located in the 5′ region near the transcription start site of genes on the same strand. Typically, a promoter is about 100 to 1000 base pairs long. Suitable promoters for use in vectors are well known in the art, such as a bacterial promoter, a viral promoter or the like. For example promoters useful in accordance without the present invention may include, but are not limited to, an immediate early cytomegalovirus (CMV) promoter, guinea pig CMV promoter, murine CMV promoter, SV40 promoter, pseudorabies virus promoters of glycoprotein X promoter, herpes simplex virus-1 alpha 4 promoter, chicken beta-actin promoter, rabbit beta-globin promoter, herpes simplex virus thymidine kinase promoter, Marek's Disease Virus promoters of glycoproteins, including any isolate or strain of MDV, such as MDV-1, MDV-2 and HVT, for example a promoter controlling expression of glycoproteins such as gC, gB, gE, or gI, infectious laryngotracheitis virus promoters such as those of glycoprotein gB, gE, gI, gD genes or any other suitable promoters.

The transcription termination region provides for efficient termination of transcription. The termination region may be from the same gene as the promoter sequence, or it may be from a different gene. It may further be from the same gene as the ORF, or it may be from a different gene.

The terms “identical” or “percent identity,” in the context of two or more nucleic acids or polypeptide sequences, refer to two or more sequences or subsequences that are the same or have a specified percentage of amino acid residues or nucleotides that are the same (i.e. , about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g. , the NCBI web site found at https://blast.ncbi.nlm.nih.gov/Blast.cgi or the like). Such sequences are then referred to as “substantially identical.” This definition also refers to, or applies to, the compliment of a particular sequence. The definition may also include sequences that have deletions, additions, and/or substitutions.

For sequence comparison, one sequence typically serves as a reference sequence, to which other sequences are compared. When using a sequence comparison algorithm, reference and comparison sequences may be entered into a computer, and sequence algorithm program parameters are selected as desired. Percent sequence identities are then generated for the comparison sequences relative to the reference sequence, based on the parameters selected. An example of an algorithm that may be suitable for determining percent sequence identity and sequence similarity are the BLAST and BLAST 2.0 algorithms, which are described in Altschul et al., (Nuc Acids Res 25: 3389-3402, 1977) and Altschul et al., (J Mol Biol 215: 403-410, 1990), respectively. BLAST and BLAST 2.0 are well known in the art and may be used to determine percent sequence identity for any nucleic acids or proteins, such as those described herein. When RNA sequences are said to be similar, or have a degree of sequence identity or homology with DNA sequences, thymidine (T) in the DNA sequence is considered equal to uracil (U) in the RNA sequence. Thus, RNA sequences are within the scope of the invention and can be derived from DNA sequences, by thymidine (T) in the DNA sequence being considered equal to uracil (U) in RNA sequences.

As used herein, a “vaccine” or an “immunogenic composition” is meant to encompass a composition suitable for administration to a subject, such as an avian subject. In general a “vaccine” is sterile, and preferably free of contaminants that are capable of eliciting an undesirable response within the subject (e.g., the compound(s) in the vaccine is pharmaceutical grade) and contains an antigen. In the present context the vaccine contains at least the recombinant Gallid herpesvirus 3 vector of the invention as antigen and possibly a further Marek's disease virus vector as described herein. Vaccines may be designed for administration to subjects in need thereof via a number of different routes of administration including in ovo, oral, intravenous, buccal, rectal, parenteral, intraperitoneal, intradermal, intracheal, intramuscular, subcutaneous, inhalational, and the like. Preferably the vaccine is administered in ovo, intramuscularly or subcutaneously. The term “vaccinating” as used herein means conferring a protective immune response and hence protecting against a disease caused by an avian pathogen.

The terms “polyvalent vaccine”, “combination or combo vaccine” and “multivalent vaccine” are used interchangeably to refer to a vaccine containing more than one antigen. The polyvalent vaccine may contain two, three, four or more antigens. The polyvalent vaccine may comprise recombinant viral vectors, active or attenuated or killed wild-type viruses, or a mixture of recombinant viral vectors and wild-type viruses in active or attenuated or killed forms.

The term “Marek's disease virus” or MDV as used herein refers to any alphaherpesvirus of the genus Mardivirus, including the herpesvirus of Turkeys (HVT), Marek's disease virus serotype 1 (MDV-1) and Gallid herpesvirus 3. Typically a MDV used as vaccine virus is Gallid herpesvirus 3 strain SB-1, naturally attenuated MDV-1 strain Rispens (CVI-988) and herpesvirus of turkeys (HVT) strain Fc126. As used herein, such a virus may include the genetic components of the virus, i.e., the genome and transcripts thereof, proteins encoded by the genome (including structural and nonstructural proteins), and functional or nonfunctional viral particles. The polynucleotide and polypeptide sequences encoding such viruses are well known in the art and would be easily found by one of skill in the art. MDV includes a wild type virus, a naturally attenuated wild type virus and a recombinant MDV. A recombinant MDV may comprise one or more heterologous polynucleotide(s) coding for and expressing at least one antigen of an avian pathogen.

The term “isolated DNA” as used herein means a DNA that has been substantially separated from, or enriched relative to, other substances with which it occurs in nature. Isolated DNA is usually at least about 80%, at least 90% pure, at least 98% pure, or at least about 99% pure, by weight and does only contain minimum amounts of contaminating DNA or protein.

The term “bacterial artificial chromosome” abbreviated as BAC as used herein refers to a DNA construct based on F-plasmid comprising an insert of about 150 to 350 kbp used for transforming and cloning in bacteria such as Escherichia coli. BAC vectors can harbor large DNA sequences, such as DNA virus genomes or DNA sequences coding for RNA virus genomes. This allows for efficient modification of viral genomes using well-established mutagenesis techniques in E. coli, such as CRISPR/Cas9, transcription activator-like effector nucleases (TALENs) or zinc-finger nucleases (ZFNs). Further encompassed is a cloning vector based on the bacterial P1-plasmid and often referred to as P1-derived artificial chromosome (PAC).

As used herein, a “pharmaceutically acceptable carrier,” “pharmaceutically acceptable adjuvant,” or “adjuvant” refers to an agent that modifies the effect of other agents and is useful in preparing a vaccine that is generally safe, non-toxic, and neither biologically nor otherwise undesirable. Such an agent may be added to a vaccine to modify the immune response of a subject by boosting the response such as to give a higher amount of antibodies and longer-lasting protection. Such an agent may include an excipient, diluent, carrier, or adjuvant that is acceptable for veterinary or pharmaceutical use. Such an agent may be non-naturally occurring, or may be naturally occurring, but not naturally found in combination with other agents in the immunogenic composition.

As used herein, an “antibody” refers to a polypeptide comprising a framework region from an immunoglobulin gene or fragments thereof that specifically binds and recognizes an antigen. The recognized immunoglobulin genes may include the kappa, lambda, alpha, gamma, delta, epsilon, and mu constant region genes, as well as the myriad immunoglobulin variable region genes. Light chains may be classified as either kappa or lambda. Heavy chains may be classified as gamma, mu, alpha, delta, or epsilon, which in turn define the immunoglobulin classes, IgY, IgG, IgM, IgA, IgD, and IgE, respectively.

### Recombinant Gallid Herpesvirus 3 (GaHV3; MDV-2) Vector

The present invention provides for a recombinant Gallid herpesvirus 3 (GaHV3; MDV-2) vector, preferably a recombinant Gallid herpesvirus 3 (GaHV3; MDV-2) strain SB-1 vector. comprising one or more heterologous polynucleotide(s) coding for and expressing at least one antigen of an avian pathogen, inserted into the intergenic loci UL3/UL4 and/or UL21/UL22 of the Gallid herpesvirus 3 vector. Preferably the vector comprises the one or more heterologous polynucleotide(s) inserted into the intergenic locus UL3/UL4 of the Gallid herpesvirus 3 vector. The heterologous polynucleotide may be incorporated anywhere within the intergenic loci UL3/UL4 and/or UL21/UL22. The intergenic locus UL3/UL4 of strain SB-1 (HQ840738.1) has the nucleotide sequence of SEQ ID NO: 1 and the intergenic locus of UL21/UL22 of strain SB-1 has the nucleotide sequence of SEQ ID NO: 2. The nucleotide sequences of the intergenic loci UL3/UL4 and UL21/UL22 are the same for strain HPRS24 (NC_002577.1). The heterologous polynucleotide may be incorporated anywhere within the sequences of SEQ ID NO: 1 or SEQ ID NO: 2, preferably within nucleotides 10 to 90 of SEQ ID NO:1 or SEQ ID NO:2, more preferably within nucleotides 20 to 80 of SEQ ID NO:1 or SEQ ID NO:2, more preferably within nucleotides 30 to 70 of SEQ ID NO:1 or SEQ ID NO:2.

The avian pathogens may be Newcastle Disease Virus (NDV), Infectious Bursal Disease Virus (i.e., IBDV or Gumboro Disease virus), Marek's Disease Virus (MDV), Infectious Laryngotracheitis Virus (ILTV), avian infectious bronchitis virus (IBV), avian encephalomyelitis virus and other picornavirus, avian reovirus, avian paramyxovirus, avian metapneumovirus, avian influenza virus, avian adenovirus, fowl pox virus, avian coronavirus, avian rotavirus, avian parvovirus, avian astrovirus and chick anemia virus, coccidiosis (Eimeria sp.), Campylobacter sp., Salmonella sp., Mycoplasma gallisepticum, Mycoplasma synoviae, Pasteurella sp., Avibacterium sp., E. coli or Clostridium sp. Preferably the antigen of an avian pathogen is an antigen of an avian virus. More preferably, the recombinant vector comprises one or more heterologous polynucleotides coding for and expressing genes from infectious bursal disease virus (IBDV), infectious laryngotracheitis virus (ILTV) Newcastle disease virus (NDV), avian influenza virus (AIV), or the like.

A particularly suitable antigen of an avian pathogen according to the invention is an antigen of an avian virus such as infectious bursal disease virus (IBDV), infectious laryngotracheitis virus (ILTV), Newcastle disease virus (NDV), avian influenza virus (AIV) or avian infectious bronchitis virus (IBV). Exemplary antigens are VP2, VP3, VP4 and VPX of IBDV; glycoprotein B, glycoprotein I, glycoprotein D, glycoprotein E and glycoprotein C of IBDV, preferably glycoprotein B, glycoprotein I or glycoprotein E; Newcastle disease virus fusion protein (NDV-F) and viral hemagglutinin neuraminidase (NDV-NH) of NDV; avian influenza hemagglutinin (HA) or neuraminidase (NA) protein of avian influenza virus, preferably of avian influenza type A virus, more preferably avian influenza A serotype H5 hemagglutinin (H5 HA), avian Influenza A serotype H7 hemagglutinin (H7 HA), avian Influenza A serotype H9 hemagglutinin (H9 HA) or avian Influenza A serotype H5N1 neuraminidase (H5N1 NA); and 51 or S2 protein of IBV. The 51 and S2 protein are glycoproteins that are cleavage products of spike glycoprotein (S glycoprotein). Particularly preferred are antigens of IBDV, more preferred is the antigen VP2 of IBDV. The at least one antigen of an avian pathogen may also be combinations of two or more of the above listed antigens.

In certain embodiments, a recombinant viral vector of the invention may have a polynucleotide encoding an IBDV viral protein or gene product, such as an IBDV VP2 protein or gene product. In another embodiment, such a recombinant viral vector may have a polynucleotide encoding an infectious laryngotracheitis virus (ILTV) viral protein or gene product, such as a ILTV gB or gC or gD or gE or gI, UL-32 protein or gene product. In another embodiment, such a recombinant viral vector may have a polynucleotide encoding a NDV viral protein or gene product, such as a NDV F or HN protein or gene product. In another embodiment, such a recombinant viral vector may have a polynucleotide encoding an Avian Influenza Virus (AIV) viral protein or gene product, such as an AIV HA or NA protein or gene product. In another embodiment, such a recombinant viral vector may have a polynucleotide encoding an infectious bronchitis virus (IBV) viral protein or gene product, such as IBV S1 or S2 protein or gene product. A polynucleotide may have more than one gene, including a gene-fusion protein or gene product, such as a NDV F-HN fusion protein, chimera, or gene product. In some embodiments, the complete coding sequence of such a gene may be used such that a full-length or fully functional protein or polypeptide is produced. Alternatively, a portion or fragment of a viral protein or polypeptide may be sufficient to provide protection against a particular virus or viruses.

In a preferred embodiment, the recombinant viral vector of the invention has a polynucleotide encoding an IBDV viral protein or gene product, such as an IBDV VP2 protein or gene product. In one embodiment the heterologous polynucleotide coding for and expressing VP2 of IBDV has at least 70%, 75%, 80%, 85%, 90%, 95%, 96%, 97%, 98% or 99% sequence similarity to a polynucleotide having the sequence as set forth in SEQ ID NO: 10, preferably has at least 70%, 75%, 80%, 85%, 90%, 95%, 96%, 97%, 98% or 99% sequence identity to a polynucleotide having the sequence as set forth in SEQ ID NO: 6 and more preferably has a sequence of SEQ ID NO: 10. In another embodiment the antigen of an avian pathogen is VP2 and has at least 70%, 75%, 80%, 85%, 90%, 95%, 96%, 97%, 98% or 99% sequence similarity to a protein having the amino acid sequence as set forth in SEQ ID NO: 12, preferably has at least 70%, 75%, 80%, 85%, 90%, 95%, 96%, 97%, 98% or 99% sequence identity to a protein having the amino acid sequence as set forth in SEQ ID NO: 12, more preferably has a protein sequence of SEQ ID NO: 12. The antigen may also be an immunogenic fragment comprising at least eight or at least ten consecutive amino acids of SEQ ID NO: 12. Moreover the immunogenic fragment or the full-length protein of IBDV VP2 may be fused to an immunogenic fragment or full-length protein of another antigen of an avian pathogen.

In another embodiment the antigen of an avian pathogen is infectious laryngotracheitis virus (Gallid herpesvirus 1) glycoprotein E (gE), glycoprotein I (gI) or glycoprotein B (gB) and has at least 70%, 75%, 80%, 85%, 90%, 95%, 96%, 97%, 98% or 99% sequence similarity to a protein having the amino acid sequence as set forth in SEQ ID NO: 31 (gE), SEQ ID NO: 32 (gI) or SEQ ID NO: 33 (gB), respectively, preferably has at least 70%, 75%, 80%, 85%, 90%, 95%, 96%, 97%, 98% or 99% sequence identity to a protein having the amino acid sequence as set forth in SEQ ID NO: 31 (gE), SEQ ID NO: 32 (gI) or SEQ ID NO: 33 (gB), respectively, more preferably has a protein sequence of SEQ ID NO: 31 (gE), SEQ ID NO: 32 (gI) or SEQ ID NO: 33 (gB), respectively. The antigen may also be an immunogenic fragment comprising at least eight or at least ten consecutive amino acids of SEQ ID NO: 31 (gE), SEQ ID NO: 32 (gI) or SEQ ID NO: 33 (gB), respectively. Moreover the immunogenic fragment or the full-length protein of ILTV gE, gI or gB may be fused to an immunogenic fragment or full-length protein of another antigen of an avian pathogen.

In another embodiment the antigen of an avian pathogen is Newcastle disease virus fusion protein (NDV-F) or Newcastle disease virus hemagglutinin neuraminidase (NDV-NH) and has at least 70%, 75%, 80%, 85%, 90%, 95%, 96%, 97%, 98% or 99% sequence similarity to a protein having the amino acid sequence as set forth in SEQ ID NO: 34 (NDV-F) or SEQ ID NO: 35 (NDV-NH), respectively, preferably has at least 70%, 75%, 80%, 85%, 90%, 95%, 96%, 97%, 98% or 99% sequence identity to a protein having the amino acid sequence as set forth in SEQ ID NO: 34 (NDV-F) or SEQ ID NO: 35 (NDV-NH), respectively, more preferably has a protein sequence of SEQ ID NO: 34 (NDV-F) or SEQ ID NO: 35 (NDV-NH), respectively. The antigen may also be an immunogenic fragment comprising at least eight or at least ten consecutive amino acids of SEQ ID NO: 34 (NDV-F) or SEQ ID NO: 35 (NDV-NH), respectively. Moreover the immunogenic fragment or the full-length protein of NDV-F or NDV-NH may be fused to an immunogenic fragment or full-length protein of another antigen of an avian pathogen.

In another embodiment the antigen of an avian pathogen is avian influenza A serotype H5 hemagglutinin (H5 HA), avian Influenza A serotype H7 hemagglutinin (H7 HA), avian Influenza A serotype H9 hemagglutinin (H9 HA) or avian Influenza A serotype H5N1 neuraminidase (H5N1 NA) and has at least 70%, 75%, 80%, 85%, 90%, 95%, 96%, 97%, 98% or 99% sequence similarity to a protein having the amino acid sequence as set forth in SEQ ID NO: 36 (H5 HA), SEQ ID NO: 37 (H7 HA), SEQ ID NO: 38 (H9 HA) or SEQ ID NO: 39 (H5N1 NA), respectively, preferably has at least 70%, 75%, 80%, 85%, 90%, 95%, 96%, 97%, 98% or 99% sequence identity to a protein having the amino acid sequence as set forth in SEQ ID NO: 36 (H5 HA), SEQ ID NO: 37 (H7 HA), SEQ ID NO: 38 (H9 HA) or SEQ ID NO: 39 (H5N1 NA), respectively, more preferably has a protein sequence of SEQ ID NO: 36 (H5 HA), SEQ ID NO: 37 (H7 HA), SEQ ID NO: 38 (H9 HA) or SEQ ID NO: 39 (H5N1 NA), respectively. The antigen may also be an immunogenic fragment comprising at least eight or at least ten consecutive amino acids of SEQ ID NO: 36 (H5 HA), SEQ ID NO: 37 (H7 HA), SEQ ID NO: 38 (H9 HA) or SEQ ID NO: 39 (H5N1 NA), respectively. Moreover the immunogenic fragment or the full-length protein of H5 HA, H7 HA, H9 HA or H5N1 NA may be fused to an immunogenic fragment or full-length protein of another antigen of an avian pathogen.

In yet another embodiment the antigen of an avian pathogen is 51 or S2 glycoprotein of IBV and has at least 70%, 75%, 80%, 85%, 90%, 95%, 96%, 97%, 98% or 99% sequence similarity to a protein having the amino acid sequence as set forth in amino acids 25 to 530 of SEQ ID NO: 40 (51) or in amino acids 553 to 1146 of SEQ ID NO: 40 (S2), respectively, preferably has at least 70%, 75%, 80%, 85%, 90%, 95%, 96%, 97%, 98% or 99% sequence identity to a protein having the amino acid sequence as set forth in amino acids 25 to 530 of SEQ ID NO: 40 (S1) or in amino acids 553 to 1146 of SEQ ID NO: 40 (S2), respectively, more preferably has a protein sequence of amino acids 25 to 530 of SEQ ID NO: 40 (S1) or amino acids 553 to 1146 of SEQ ID NO: 40 (S2), respectively. The antigen may also be an immunogenic fragment comprising at least eight or at least ten consecutive amino acids of amino acids 25 to 530 of SEQ ID NO: 40 (S1) or amino acids 553 to 1146 of SEQ ID NO: 40 (S2), respectively. Moreover the immunogenic fragment or the full-length protein of S1 or S2 protein may be fused to an immunogenic fragment or full-length protein of another antigen of an avian pathogen.

The antigens suitable in the present invention may also be an allelic variant of any of the antigens of an avian pathogen disclosed herein. The term “allelic variant” refers to a polynucleotide or a polypeptide containing polymorphisms that lead to changes in the amino acid sequences of a protein and that exist within a natural population (e.g., a virus species or variety). Such natural allelic variations can typically result in 1-5% variance in a polynucleotide or a polypeptide. Allelic variants can be identified by sequencing the nucleic acid sequence of interest in a number of different species, which can be readily carried out by using hybridization probes to identify the same gene genetic locus in those species. Any and all such nucleic acid variations and resulting amino acid polymorphisms or variations that are the result of natural allelic variation and that do not alter the functional activity of gene of interest, are intended to be within the scope of the invention.

The polynucleotides used in the present invention may be codon optimized for a specific host. “Codon optimized” as used herein refers to a polynucleotide that is genetically engineered to increase its expression in a given species. To provide optimized polynucleotides coding for IBDV VP2 polypeptides, the DNA sequence of the VP2 protein gene can be modified to 1) comprise codons preferred by highly expressed genes in a particular species; 2) comprise an A+T or G+C content in nucleotide base composition to that substantially found in said species; 3) form an initiation sequence of said species; or 4) eliminate sequences that cause destabilization, inappropriate polyadenylation, degradation and termination of RNA, or that form secondary structure hairpins or RNA splice sites. Increased expression of VP2 protein in said species can be achieved by utilizing the distribution frequency of codon usage in eukaryotes and prokaryotes, or in a particular species.

The viral antigens coded for and expressed by the recombinant viral vector of the present invention may be encoded by a viral gene. However, one of skill in the art will appreciate in this regard that it may not be required to incorporate the entirety of a particular viral gene in order to obtain a desired immune response. Rather, a portion of such a gene may be sufficient. Optimization of a desired viral protein or polynucleotide encoding such a protein regardless of the length of the protein may be readily carried out using methods known in the art. One of skill in the art will further appreciate that modifications may be made to a viral gene or genes, or the proteins encoded thereby, to increase the immunogenicity of the viral protein when introduced into the subject.

Moreover, the recombinant Gallid herpesvirus 3 vector may comprise more than one heterologous polynucleotide coding for and expressing at least one antigen of an avian pathogen. In one embodiment the vector of the invention comprises two or three heterologous polynucleotides, preferably two. Preferably at least one of the heterologous polynucleotides codes and expresses IBDV VP2 as described above.

Thus, in some embodiments, the recombinant viral vector may express one heterologous protein from a single virus species or may express more than one heterologous protein from a single virus species different to Gallid herpesvirus 3 in order to obtain protection against MDV and a further disease caused by an avian virus. For instance, in one embodiment, the invention provides a recombinant viral vector comprising the GaIHV3 genome and a polynucleotide coding for and expressing an antigen of a different pathogen, thus providing protection in an avian species such as poultry against Marek's disease, and at least one other disease caused by a different avian pathogen. Specifically the invention provides a recombinant viral vector comprising the GaIHV3 genome and a polynucleotide coding for and expressing an antigen of a different virus, thus providing protection in an avian species such as poultry against Marek's disease, and at least one disease caused by a different avian viral pathogen. For example, the recombinant viral vector in accordance with the invention may provide protection in avian species such as poultry against MDV and IBDV, or may provide protection against MDV and ILTV, or may provide protection against MDV and NDV, or may provide protection against MDV and AIV, or may provide protection against MDV and IBV. Preferably, the recombinant viral vector in accordance with the invention may provide protection in avian species such as poultry against MDV and IBDV

In other embodiments, the recombinant viral vector may express heterologous proteins from more than one virus species in order to obtain protection to multiple viruses in addition to MDV. For instance, in one embodiment, the invention provides a recombinant viral vector comprising the GaIHV3 genome and at least two polynucleotides coding for and expressing an antigen of two different pathogens, thus providing protection in an avian species such as poultry against Marek's disease, and at least two other diseases caused by avian pathogens. Specifically the invention provides a recombinant viral vector comprising the GaHV3 genome and at least two heterologous polynucleotides coding for and expressing antigens of two different viruses, thus providing protection in avian species such as poultry against Marek's disease, and at least two other disease caused by avian viral pathogen. For example, the recombinant viral vector in accordance with the invention may provide protection in avian species such as poultry against MDV, IBDV and one of the diseases selected from NDV, AIV and IBV; or against MDV, NDV and one of the diseases selected from IBDV, AIV and IBV; or against MDV, AIV and one of the diseases selected from IBDV, NDV and IBV; or against MDV, IBV and one of the diseases selected from IBDV, NDV and AIV. Preferably, the recombinant viral vector in accordance with the invention may provide protection in avian species such as poultry against MDV, IBDV and one of the diseases selected from NDV, AIV and IBV.

In case the recombinant viral vector comprises more than one heterologous polynucleotide, the polynucleotides may be inserted at the same insertion site or at different insertion sites. Preferably at least one of the heterologous polynucleotides is inserted into the intergenic locus of UL3/UL4. Further the more than one heterologous polynucleotide may be coded for and expressed by the same expression cassette using one promoter or by separate expression cassettes.

Thus, in accordance with the invention, the recombinant viral vector may comprise one or more transgene(s) operatively linked to one or more promoters for expression of one or more viral proteins or peptides or fragments or portions thereof. In some embodiments, a single transgene may be operably linked to a single promoter, or more than one transgene may be operatively linked to a single promoter. In other embodiments, more than one transgene may be present in a recombinant vector wherein a first transgene is operatively linked to a first promoter and a second transgene is operatively linked to a second promoter.

Successful expression of the inserted cDNA genetic sequence by the modified infectious virus requires two conditions. First, the insertion must be into a non-essential region of the genome of the virus in order for the modified virus to remain viable (intact replication and amplification), e.g., by insertion into the intergenic loci UL3/UL4 and/or UL21/UL22. The second condition for expression of inserted cDNA is the presence of a regulatory sequences allowing expression of the gene in the viral background (for instance: promoter, enhancer, donor and acceptor splicing sites and intron, Kozak translation initiation consensus sequence, polyadenylation signals, untranslated sequence elements).

In general, it is advantageous to employ a strong promoter functional in eukaryotic cells. The promoters include, but are not limited to, an immediate early cytomegalovirus (CMV) promoter, guinea pig CMV promoter, murine CMV promoter an SV40 promoter, pseudorabies virus promoters such as that of glycoprotein X promoter, herpes simplex virus-1 such as the alpha 4 promoter, chicken beta-actin promoter, rabbit beta-globin promoter, herpes simplex virus thymidine kinase promoter, Marek's Disease Viruses (including MDV-1, MDV-2 and HVT) promoters such as those driving glycoproteins gC, gB, gE, or gI expression, infectious laryngotracheitis virus promoters such as those of glycoprotein gB, gE, gI, gD genes, or other herpesvirus promoters. When the insertion locus consists of a SB-1 gene (for instance, gC, gD, US2 or US 10 genes), the foreign gene can be inserted into the vector with no additional promoter sequence since the promoter of the deleted gene of the vector will drive the transcription of the inserted foreign gene.

### An Intermediate Recombinant Gallid Herpesvirus 3 (GaHV3; MDV-2) Vector Comprising One or More Maker(s)

In a further aspect the invention relates to an intermediate product used for the production of the recombinant Gallid herpesvirus 3 of the invention. This intermediate recombinant Gallid herpes virus 3 vector comprises one or more marker(s) inserted into the intergenic loci UL3/UL4 and/or UL21/UL22 of the Gallid herpesvirus 3 vector, wherein the recombinant Gallid herpesvirus 3 vector is preferably a Gallid herpesvirus 3 strain SB-1 vector. Preferably the one or more marker(s) is/are inserted into the intergenic locus of UL3/UL4.

The marker may be a selection marker gene, a reporter gene or a DNA bar code. Selection markers may encode for example, biocide resistance, or antibiotic resistance (e.g., kanamycin, G418, bleomycin, hygromycin, etc.). A preferred selectable marker is E. coli galactokinase gene K (galK), which confers sensitivity do 2-desoxy-galactose (2-DOG), but allows survival and growth on galactose as the only carbon source. Another preferred selectable marker is kan/sacB, an expression cassette containing the neomycin (kanamycin) gene from Tn5 and the sacB gene from Bacillus subtilis. Selectable markers are well known to one of skill in the art and may include any markers suitable for use in accordance with the invention. Reporter genes may be used to monitor expression, but usually do not result in death of a cell. Suitable reporter genes include for example, a β-glucuronidase or uidA gene (GUS), one or more of the various fluorescent protein genes, such as green fluorescent protein (GFP), red fluorescent protein (RFP), or any one of a large family of proteins which fluorescence at characteristic wavelengths, a β-lactamase gene, a gene that encodes an enzyme for which various chromogenic substrates are known, a luciferase gene, a xyIE gene, which encodes a catechol dioxygenase that converts chromogenic catechols, an oc-amylase gene, a tyrosinase gene, which encodes an enzyme capable of oxidizing tyrosine to DOPA and dopaquinone, which in turn condense to melanin, or an a-galactosidase, which catalyzes a chromogenic a-galactose substrate. A DNA bar code does not have a specific function other than having a detectable and unique sequence. Preferably the marker is a selection marker gene or a reporter gene. More preferably the intermediate recombinant Gallid herpesvirus 3 vector comprises one or more expression cassette(s) comprising a selection marker gene and/or a reporter gene. In a preferred embodiment the selection marker gene or reporter gene is galK or a kan/sacB combination. The marker may serve as a placeholder at the intergenic loci of UL3/UL4 and/or UL21/UL22 for the at least one antigen of an avian pathogen, as it can be easily replaced by the at least one antigen of an avian pathogen as described below.

### Methods for Producing a Recombinant Gallid Herpesvirus 3 (GaHV3; MDV-2) Vector

The present invention further provides a method for producing a recombinant Gallid herpesvirus 3 vector comprising the introduction of one, two or more polynucleotides into the intergenic loci UL3/UL4 and/or UL21/UL22 of the Gallid herpesvirus 3 vector, wherein the Gallid herpesvirus 3 vector is preferably a Gallid herpesvirus 3 strain SB-1 vector.

In one embodiment the method of producing a recombinant Gallid herpesvirus 3 vector comprises (a) providing a Gallid herpesvirus 3 vector, (b) inserting one or more heterologous polynucleotide(s) coding for and expressing at least one antigen of an avian pathogen into the intergenic loci UL3/UL4 and/or UL21/UL22 of the Gallid herpesvirus 3 vector, and optionally (c) amplifying the Gallid herpesvirus 3 vector comprising one or more heterologous polynucleotide(s) coding for at least one antigen of an avian pathogen of step (b).

In another embodiment the method of producing a recombinant Gallid herpesvirus 3 vector comprises (a) providing a Gallid herpesvirus 3 vector comprising one or more marker(s) in the intergenic loci UL3/UL4 and/or UL21/UL22 of the Gallid herpesvirus 3 vector, (b) replacing the one or more marker(s) with an expression cassette comprising one or more heterologous polynucleotides coding for at least one antigen of an avian pathogen, and (c) amplifying the Gallid herpesvirus 3 vector comprising one or more heterologous polynucleotide(s) coding for at least one antigen of an avian pathogen of step (b). The recombinant Gallid herpes virus 3 may be coded for and expressed by a bacterial artificial chromosome (BAC) or a P1-derived artificial chromosome (PAC), preferably a BAC. Thus, step (a) may comprise providing a bacterial artificial chromosome or a P1-derived artificial chromosome comprising a polynucleotide coding for the (recombinant) Gallid herpesvirus 3 vector. The skilled person would understand that the advantage of using BAC or PAC as a vector for the Gallid herpesvirus 3 is that the insertion of the one or more heterologous polynucleotide(s) and the amplification can be performed in E. coli. This also includes a screening step for the desired viral vector. The method may therefore further comprise the steps of (d) isolating the BAC or PAC comprising a polynucleotide coding for the recombinant Gallid herpesvirus 3 vector comprising one or more heterologous polynucleotide(s) coding for at least one antigen of an avian pathogen of step (b) and (e) transfecting chicken embryonic fibroblasts with the BAC or PAC of step (d).

As outlined above the term “amplifying the Gallid herpes virus 3 vector” relates to amplification in E. coli or in avian host cells such as chicken embryonic fibroblasts, depending on the Gallid herpes virus 3 vector used. In E. coli this involves culturing E. coli transfected with the plasmid (BAC or PAC) coding for the desired viral vector under suitable condition and isolating the plasmid DNA. In avian cells, the cells containing the desired viral vector are cultured and either the supernatant or whole cells containing the desired viral vector are harvested. For propagation of the virus, the harvested desired viral vector may be used directly for transducing avian host cells or may be frozen and stored before further use.

The term “replacing the one or more marker(s)” as used herein means inserting an expression cassette at the site of the one or more marker(s) by any methods for targeted integration known to the person skilled in the art. E.g., the expression cassette comprising one or more heterologous polynucleotide coding for at least one antigen of an avian pathogen may be inserted by homologous recombination. Alternatively the one or more marker(s), preferably an expression cassette coding for the one or more marker(s), may be flanked by recognition sites (e.g., loxP or FRT sites) for a site specific recombinase (e.g., Cre or Flp recombinase) and one or more marker(s) are then replaced by an expression cassette comprising one or more heterologous polynucleotide coding for at least one antigen of an avian pathogen, wherein the expression cassette is also flanked by recognition sites for the same site specific recombinase using Cre-loxP recombination or Flp-FRT recombination technology.

A recombinant Gallid herpesvirus-3 (MDV-2) viral vector may be constructed as described in WO 2013/057235 and WO 2017/027324 in two steps. First, the Gallid herpesvirus-3 (MDV-2) genomic regions flanking the locus of insertion are cloned into an E. coli plasmid construct; unique(s) restriction site(s) is (are) placed between the two flanking regions (insertion plasmid) in order to allow the insertion of the donor expression cassette DNA. Separately, the cDNA or DNA gene sequence to be inserted is preceded by a promoter region (gene start region) and a terminator (or poly-adenylation, poly-A) sequence which is specific for the Gallid herpesvirus-3 (MDV-2) vector and/or eukaryotic cells, such as mammalian cells. The whole expression cassette (promoter-transgene-poly-A) is then cloned into the unique(s) restriction site(s) of the insertion plasmid to construct the “donor plasmid” which contains the expression cassette flanked by Gallid herpesvirus-3 (MDV-2) “arms” flanking the insertion locus. The resulting donor plasmid construct is then amplified in E. coli and plasmid DNA is extracted. The plasmid is then linearized using a restriction enzyme that cuts the plasmid backbone (outside the Gallid herpesvirus-3 (MDV-2) arms and expression cassette). Chicken embryo fibroblasts are then co-transfected with parental Gallid herpesvirus-3 (MDV-2) DNA and linearized donor plasmid DNA. The resulting virus population is then cloned by multiple limiting dilution steps where recombinant viral vector expressing the transgene is isolated from the non-expressing viral population. Similarly, another foreign cassette can be inserted in another locus of insertion to create a double Gallid herpesvirus-3 (MDV-2) vector expressing two recombinant genes (polynucleotides). The second cassette can also be inserted into the same locus. The recombinant Gallid herpesvirus-3 (MDV-2) is produced in primary chicken embryo fibroblasts similarly to the parental Gallid herpesvirus-3 (MDV- 2) strain SB-1 MD vaccine. After incubation, infected cells are harvested, mixed with a freezing medium allowing survival of infected cells, and frozen usually in cryovial or glass ampoules and stored in liquid nitrogen.

Alternatively, a recombinant Gallid herpesvirus-3 (MDV-2) vector may be constructed as bacterial artificial chromosome (BAC) or as P1-derived artificial chromosome (PAC). Specifically, a pSB-1 BAC clone (Petherbridge Let al., J Virol Methods. 2009; 158: 11-7) comprising the entire Gallid herpesvirus 3 strain SB-1 genome (Genebank Accession Number HQ840738.1 may be used to generate a recombinant virus with a galK expression cassette (SEQ ID NO: 8) inserted into the indicated locus. The primers (such as the primers listed in Table 2) have homologous sequences for the exact nucleotides of the region where the fragment is going to be inserted. Homologous recombination between the homologous sequences from the primers and the nucleotide sequence of the locus in the vector results in the insertion of the fragment. Positive colonies are selected based on their ability to utilise galactose as the sole carbon source in a minimal media. The galK expression cassette (SEQ ID NO: 8) may than be replaced by the VP2 expression cassette (SEQ ID NO: 9) or any other heterologous polynucleotide expression cassette. Positive colonies are selected based on their ability to grow in the presence of 2-deoxy-galactose and the integration of the VP2 expression cassette is confirmed by specific PCR and sequencing. Recombinant Gallid herpesvirus vector according to the invention is isolated from E. coli and CEF are transfected with the BAC DNA encoding the recombinant Gallid herpesvirus-3 vector of the invention and reconstituted viruses are passaged to generate working virus stocks.

### Vaccines

In some aspects the recombinant Gallid herpesvirus 3 vector comprising one or more heterologous polynucleotide according to the invention may be used as a vaccine, i.e., an immunogenic composition, for administering to a subject, such as a chicken or other poultry in order to provide protection from one or more avian viruses. Thus, the vaccine comprises a recombinant Gallid herpesvirus 3 (GaHV3; MDV-2) vector comprising one or more heterologous polynucleotide(s) coding for and expressing at least one antigen of an avian pathogen, inserted into the intergenic loci UL3/UL4 and/or UL21/UL22 of the Gallid herpesvirus 3 vector, wherein the recombinant Gallid herpesvirus 3 vector is preferably a recombinant Gallid herpesvirus 3 strain SB-1 vector.

The vaccine of the present invention may comprise a recombinant Gallid herpesvirus-3 (MDV-2) vector of the present invention and a pharmaceutically or veterinarily acceptable carrier, excipient or adjuvant.

In another embodiment, the vaccine comprises: i) a recombinant Gallid Herpesvirus-3 (MDV-2) vector comprising one or more heterologous polynucleotides coding for and expressing at least one antigen of an avian pathogen inserted into the intergenic loci UL3/UL4 and/or UL21/UL22 of the Gallid herpesvirus 3 vector, wherein the recombinant Gallid herpesvirus 3 vector is preferably a recombinant Gallid herpesvirus 3 strain SB-1 vector; and ii) at least a further Marek's disease virus (MDV) vector. The further MDV vector is preferably selected from the group consisting of Gallid herpesvirus 3 vector, naturally attenuated MDV-1 strain Rispens (CVI-988) vector and herpesvirus of turkeys (HVT or MDV-3) strain Fc126 vector, more preferably from Gallid herpesvirus 3 strain SB-1 vector, naturally attenuated MDV-1 strain Rispens (CVI-988) vector and herpesvirus of turkeys (HVT or MDV-3) strain Fc126 vector. The further MDV vector may be a wild type vector, preferably selected from the group consisting of Gallid herpesvirus 3 vector, naturally attenuated MDV-1 strain Rispens (CVI-988) vector and herpesvirus of turkeys (HVT or MDV-3) strain Fc126 vector or a recombinant MDV vector preferably derived from a MDV vector selected from the group consisting of Gallid herpesvirus 3 vector, naturally attenuated MDV-1 strain Rispens (CVI-988) vector and herpesvirus of turkeys (HVT or MDV-3) strain Fc126 vector.

The recombinant Marek's disease virus (MDV) vector may comprise one or more heterologous polynucleotide(s) coding for and expressing at least one antigen of an avian pathogen. The recombinant MDV vector can be derived from the naturally attenuated MDV-1 strain Rispens (CVI-988) vector or the herpesvirus of turkeys (HVT or MDV-3) strain Fc126 vector. The recombinant MDV vector may also be a second recombinant Gallid herpesvirus 3 vector, preferably a recombinant Gallid herpesvirus 3 strain SB-1 vector, in addition to the first recombinant Gallid herpesvirus 3 vector of the invention. The person skilled in the art would understand that the second recombinant Gallid herpesvirus 3 vector is different to the first recombinant Gallid herpesvirus 3 vector in that they express different heterologous polynucleotides. Thus, the second recombinant Gallid herpesvirus 3 vector comprises at least one heterologous polynucleotide coding for and expressing a different antigen of an avian pathogen as the one or more heterologous polynucleotides of the first recombinant Gallid herpesvirus 3 vector. The person skilled in the art would further understand that the two recombinant Gallid herpesvirus 3 vectors can express different heterologous polynucleotides and hence may confer protection to different diseases caused by an avian pathogen in addition to Marek's disease. This second recombinant Gallid herpesvirus 3 vector may likewise comprise the one or more heterologous polynucleotide(s) inserted into the intergenic loci UL3/UL4 and/or UL21/UL22 of said second Gallid herpesvirus 3 vector, preferably into the intergenic locus UL3/UL4. While the first and the second recombinant Gallid herpesvirus 3 vector are preferably recombinant Gallid herpesvirus 3 strain SB-1 vectors, they may also be derived of different strains such as the first vector being derived from a SB-1 strain and the second vector from a HPRS24 strain or the first vector being derived from a HPRS24 strain and the second from a HPRS24 strain. Preferably the (first) recombinant Gallid herpesvirus 3 vector and the further Marek's disease virus as described above are administered together in one dosage form. Alternatively the (first) recombinant Gallid herpesvirus 3 vector and the further Marek's disease virus as described above may be administered in separate dosage form at the same time or at different time points.

The vaccine according to the invention is designed to generate antibody immunity and/or cellular immunity in a subject. The vaccine may further comprise a pharmaceutically acceptable excipient, carrier or adjuvant. A pharmaceutically acceptable excipient, carrier or adjuvant may be a substance that enhances an immune response in a subject to an exogenous antigen, including but not limited to, adjuvants, liposomes, biodegradable microspheres. A pharmaceutically acceptable carrier or adjuvant may contain a substance designed to protect the antigen from rapid catabolism, such as aluminum hydroxide or mineral oil, or a stimulator of immune responses, such as proteins derived from Bordetella pertussis or Mycobacterium tuberculosis. The vaccines according to the invention may comprise or consist essentially of one or more adjuvants. Suitable adjuvants for use in the practice of the present invention are (1) polymers of acrylic or methacrylic acid, maleic anhydride and alkenyl derivative polymers, (2) immunostimulating sequences (ISS), such as oligodeoxyribonucleotide sequences having one or more non-methylated CpG units (Klinman et al, 1996; W098/16247), (3) an oil in water emulsion, such as the SPT emulsion described on p 147 of “Vaccine Design, The Subunit and Adjuvant Approach” published by M. Powell, M. Newman, Plenum Press 1995, and the emulsion MF59 described on p 183 of the same work, (4) cation lipids containing a quaternary ammonium salt, e.g., DDA (5) cytokines, (6) aluminum hydroxide or aluminum phosphate, (7) saponin or (8) other adjuvants discussed in any document cited and incorporated by reference into the instant application, or (9) any combinations or mixtures thereof. Commercially available adjuvants may include for example, Freund's Incomplete Adjuvant and Complete Adjuvant, Merck Adjuvant 65, aluminum salts such as aluminum hydroxide gel (alum) or aluminum phosphate; CpG oligonucleotides, salts of calcium, iron or zinc; an insoluble suspension of acylated tyrosine; acylated sugars; cationically or anionically derivatized polysaccharides; polyphosphazenes; biodegradable microspheres; and monophosphoryl lipid A.

One of skill in the art will be able to identify appropriate pharmaceutically acceptable carriers for use with the present invention. A suitable pharmaceutically acceptable carrier may be determined in part by the particular composition being administered, as well as by the particular method used to administer the composition. Accordingly, a wide variety of suitable formulations of vaccines are available that may be of use in the present invention. A suitable pharmaceutically acceptable carrier includes aqueous and non-aqueous, isotonic sterile injection solutions, which can contain antioxidants, buffers, bacteriostats, and solutes that render the formulation isotonic with the blood of the intended subject, and aqueous and non-aqueous sterile suspensions that can include suspending agents, solubilizers, thickening agents, stabilizers, and preservatives. The pharmaceutically acceptable carrier or excipients may be any compound or combination of compounds facilitating the administration of the vector (or protein expressed from an inventive vector in vitro), or facilitating transfection or infection and/or improve preservation of the vector (or protein). Pharmaceutically acceptable carrier or excipients that can be used for methods of this invention include, but are not limited to, 0.9% NaCl (e.g., saline) solution or a phosphate buffer, poly-(L-glutamate) or polyvinylpyrrolidone. Such vaccines may also comprise buffers (e.g. , neutral buffered saline or phosphate buffered saline), carbohydrates (e.g. , glucose, mannose, sucrose or dextrans), mannitol, proteins, polypeptides or amino acids such as glycine, antioxidants, bacteriostats, chelating agents such as EDTA or glutathione, adjuvants (e.g. , aluminum hydroxide), solutes that render the formulation isotonic, hypotonic, or weakly hypertonic with the blood of a subject, suspending agents, thickening agents, and/or preservatives. Alternatively, compositions of the present invention may be formulated as a lyophilizate. Injection solutions and suspensions may be prepared from sterile powders, granules, and tablets.

Administration may be in any convenient manner, e.g., by injection, oral administration, inhalation, transdermal application, or rectal administration. Administration may also be by injection in ovo. Injection of a recombinant viral vector or a vaccine as described herein may be provided to a subject such as poultry in a single administration or dose, or may be administered more than once, such as in repeated doses.

A vaccine may generally be used for prophylactic and/or therapeutic purposes. For example, in accordance with the invention, the vaccine may be provided to a subject, such as an avian species, to vaccinate against one or more diseases caused by one or more avian pathogens. Typically the vaccine is administered prior to infection with or exposure to an avian pathogen in order to provide protection against infection with one or more avian pathogen or development of clinical symptoms caused by one or more avian pathogen. Preferably the avian pathogen is an avian virus, more preferably the avian pathogen is Marek's disease virus and one or more further avian virus. The one or more further avian viruses are preferably infectious bursal disease virus (IBDV), infectious laryngotracheitis virus (ILTV), Newcastle disease virus (NDV), avian influenza virus or avian infectious bronchitis virus (IBV). In order to provide protection or vaccinate against an avian pathogen the vaccine is administered at an early age, preferably in ovo, such as in 18 d old embryonated eggs, such as in chicken eggs, or in chicks, preferably in 1 day old chicks. In another embodiment, the vaccine may be also provided to a subject, such as a bird, after infection with or exposure to one or more avian pathogen in order to provide treatment of the pathogen in the subject, such as by reducing or eliminating infection in the subject.

Vaccines according to the invention may be provided in single-dose or multi-dose containers, such as sealed ampoules or vials. Such containers may be sealed to preserve sterility of the composition until use. In general, compositions as described herein may be stored as suspensions, solutions, or emulsions in oily or aqueous vehicles. Alternatively, such a composition may be stored in a freeze-dried condition requiring only the addition of a sterile liquid carrier immediately prior to use.

The dose administered to a subject in the context of the present invention should be sufficient to affect a beneficial prophylactic response in the subject over time, such as protecting a subject, such as a chicken or other poultry, against clinical symptoms caused by one or more avian pathogens or vaccinating against one or more diseases caused by one or more avian pathogens. A protective dose typically has about 3000 to 5000 pfu of the recombinant Gallid herpesvirus 3 of the invention.

### Use of the Vaccine

Another aspect of the invention relates to a method for inducing an immunological response in an animal against one or more antigens or a protective response in an animal against one or more avian pathogens, which method comprises inoculating the animal at least once with the vaccine of the present invention. Yet another aspect of the invention relates to a method for inducing an immunological response in an animal to one or more antigens or a protective response in an animal against one or more avian pathogens in a prime-boost administration regimen, which is comprised of at least one primary administration and at least one booster administration using at least one common polypeptide, antigen, epitope or immunogen. The vaccine used in primary administration may be same or may be different from those used as a booster.

Specifically the vaccine of the present invention is used for vaccinating an avian species against one or more diseases caused by one or more avian pathogens preferably against Marek's disease and one or more diseases caused by one or more avian pathogens. In some embodiments the vaccine of the present invention is for use in protecting an avian species against clinical symptoms caused by one or more avian pathogens, preferably against clinical symptoms caused by Marek's disease virus and clinical symptoms caused by one or more avian pathogens.

Also provided is a method of treating an avian species for protection against Marek's Disease and one or more diseases caused by one or more avian pathogens comprising the step of administering an effective amount of the vaccine according to the invention.

The animal to be vaccinated is an avian species and typically poultry. Suitable animals are, e.g., turkey, chicken, quail, ducks geese or pigeons. Preferably the animal to be vaccinated is turkey or chicken, more preferably chicken.

The one or more avian pathogens causing the disease or clinical symptoms is preferably an avian pathogen selected from the group consisting of Newcastle disease virus (NDV), infectious bursal disease virus (IBDV), avian infectious laryngotracheitis virus (ILTV), avian influenza virus (AIV) or avian infectious bronchitis virus (IBV).

Other suitable avian pathogens are avian encephalomyelitis virus and other picornavirus, avian reovirus, avian paramyxovirus, avian metapneumovirus, avian adenovirus, fowl pox virus, avian coronavirus, avian rotavirus, avian parvovirus, avian astrovirus and chick anemia virus, coccidiosis (Eimeria sp.), Campylobacter sp., Salmonella sp., Mycoplasma gallisepticum, Mycoplasma synoviae, Pasteurella sp., Avibacterium sp., E. coli or Clostridium sp. Preferably the avian pathogen is an avian virus.

Preferred diseases vaccinated or protected against that are caused by an avian pathogen are Newcastle disease (ND) caused by Newcastle disease virus (NDV), infectious bursal disease (IBD) caused by infectious bursal disease virus (IBDV), avian infectious laryngotracheitis (ILT) caused by avian infectious laryngotracheitis virus (ILTV), avian influenza caused by avian influenza virus (AIV) or avian infectious bronchitis (IB) caused by avian infectious bronchitis virus (IBV).

Usually, one administration of the vaccine is performed either in ovo or in chicks. Chicks are preferably administered at one day-of-age by the subcutaneous or intramuscular route. In ovo administration is typically performed in 17-19 day-old embryonated eggs, preferably in 18 day-old embryonated eggs, e.g., from chicken. The animals are preferably at least 17-day-embryo or one day old at the time of the first administration. A second administration can be done within the first 10 days of age.

In one embodiment the recombinant Gallid herpesvirus 3 (GaHV3; MDV-2) vector or the vaccine of the invention is used for protecting an avian species against clinical symptoms caused by infectious bursal disease virus. The skilled person would understand that the at least one antigen of an avian pathogen coded for and expressed by one or more heterologous polynucleotides would be an antigen of infectious bursal disease virus, preferably VP2 of infectious bursal disease virus.

A variety of administration routes in day-old chicks may be used such as subcutaneously or intramuscularly, intradermally, transdermally. The in ovo vaccination can be performed in the amniotic sac and/or the embryo. Commercially available in ovo and s.c. administration devices can be used for vaccination.

### EXAMPLES

### Cloning of galK Expression Cassette Into SB-1 Virus Genome

The pSB-1 BAC clone (Petherbridge Let al., J Virol Methods. 2009; 158: 11-7) comprising the entire Gallid herpesvirus 3 strain SB-1 genome (GenBank Accession Number: HQ840738.1) was used to generate different recombinant viruses with a galK expression cassette (SEQ ID NO: 8) inserted into the indicated locus by employing a homologous recombination based technique using recombination proteins provided from lambda phage. Briefly, galK expression cassette was amplified by specific primers (Table 2) tagged for the specific locus where the insertion is going to occur. E. coli strain SW102 harboring pSB-1 BAC was transformed with the amplified fragment. With the aid of homologous recombination, the fragment was inserted into the exact location directed by the homologous sequences (tags). For example primer pair UL3/4F-GalKF and UL3/4R-GalKR tag the galK cassette with UL3/4 sequence, so that the fragment will be inserted into the UL3/UL4 region by homologous recombination. Positive colonies were selected based on their ability to utilize galactose as the sole carbon source in a minimal media. Protocols for the galK selection-based recombineering approach have been described (Zhao Y, Nair V. Mutagenesis of the Repeat Regions of Herpesviruses Cloned as Bacterial Artificial Chromosomes. In: Braman J, editor. In Vitro Mutagenesis Protocols. 3rd ed: Humana Press; 2010. p. 53-74; Warming S et al., Nucleic Acids Res. 2005; 33:e36). The integration of the galK expression cassette was confirmed by specific PCR and sequencing.

Exemplary insertion sites of UL3/4, UL10/11 and UL21/22 are shown below. The position of the exact insertion site is shown in bold, highlighting the two nucleotides 5′ and 3′ of the insertion site. The numbers at the 5′ and 3′ end of the sequences shown in Table 1 as well as the numbers indicated for the insertion site refer to the Gallid herpesvirus 3 strain SB-1 genome (GenBank accession number HQ840738.1).

TABLE 1
Insertion loci

| In- | SEQ | | |
|---|---|---|---|
| sertion | ID | | Insertion |
| locus | NO | Sequence | site |
| UL3/4 | 1 | 19456-caagaagcatctaaaacg | 19516- |
| | | cgcttgattgtcgagtggctgaat | insertion- |
| | | aaaatctttattgatcgactcgct | 19517 |
| | | ttcctatttctgatttaataacca | (between |
| | | tagatg-19551 | nucleotides |
| | | | 61 and 62 of |
| | | | SEQ ID NO: 1) |

TABLE 2
Primers used for generating the galK expression cassette

| Primer | Sequence | SEQ ID NO: |
|---|---|---|
| UL21/22F- | gccgggcatatacagaacgtaagccaagctggagtttgtgt | SEQ ID NO: 13 |
| GalKF | aagtatgtgcctgttgacaattaatcatcggca | |
| UL21/22R- | CCTGTACTGTTGTGTTTATTCGCGAACCGCCCTTCCCGCAC | SEQ ID NO: 14 |
| GalKR | GCTGTAGAGTCAGCACTGTCCTGCTCCTTG | |
| UL26/27F- | ggatccgctatgtcgacgtataagtttatacattttgcgac | SEQ ID NO: 15 |
| GalKF | cgcaatagccctgttgacaattaatcatcggca | |
| UL26/27R- | TCTCGATAAATAAATTCTCACGTGCGCATACGATTATTTTA | SEQ ID NO: 16 |
| GalKR | CTTTTATTTTCAGCACTGTCCTGCTCCTTG | |
| UL3/4F- | taaaacgcgcttgattgtcgagtggctgaataaaatcttta | SEQ ID NO: 17 |
| GalKF | ttgatcgaccctgttgacaattaatcatcggca | |
| UL3/4R- | ATAGATTCCCCGCCCCATCTATGGTTATTAAATAGAAATAG | SEQ ID NO: 18 |
| GalKR | GAAAGCGATCAGCACTGTCCTGCTCCTTG | |
| UL10/11F- | agtgaatgggatgaataggaacgcccgaaacataataaaac | SEQ ID NO: 19 |
| GalKF | gctaaatctcctgttgacaattaatcatcggca | |
| UL10/11R- | AATGTAACGATACGTTATAGTGATAAATAAGTCGCGCGACA | SEQ ID NO: 20 |
| GalKR | ATCACTTGTTCAGCACTGTCCTGCTCCTTG | |
| UL40/41F- | gtatcgaacgatctttaattagcctgtgtgcaactgtactt | SEQ ID NO: 21 |
| GalKF | tctacccctcctgttgacaattaatcatcggca | |
| UL40/41R- | ACACGGTGCGAAAAGCGATAATTTGAATTCATTTATTAATT | SEQ ID NO: 22 |
| GalKR | CTTGTGGGTTCAGCACTGTCCTGCTCCTTG | |
| UL50/51F- | gaaacggcagcattcgatacggaatcgggcaggagcgagag | SEQ ID NO: 23 |
| GalKF | agagtgtgccctgttgacaattaatcatcggca | |
| UL50/51R- | GTATGTAATCGTGCGCAACTATACATTATTGCCCGCTCGAC | SEQ ID NO: 24 |
| GalKR | CCGAAGCCGTCAGCACTGTCCTGCTCCTTG | |

### Cloning of IBDV VP2 Expressing Cassette Into SB-1 Virus Genome

The pSB-1 BAC clones comprising a galK expression cassette (SEQ ID NO: 8) in the UL3/4 (SEQ ID NO: 1), UL21/22 (SEQ ID NO: 2) or UL10/11 (SEQ ID NO:3) intragenic locus of the SB-1 virus genome were used to generate three different recombinant viruses pSB-1-UL3/4VP2, pSB-1-UL21/22VP2 and pSB-1-UL10/11VP2 that contain the IBDV VP2 expression cassette (SEQ ID NO:9). The galK expression cassette (SEQ ID NO: 8) was replaced by the VP2 expression cassette (SEQ ID NO: 9) amplified from the recombinant HVT expressing IBDV VP2 (Darteil R et al., Virology 1995; 211: 481-90) comprising a nucleotide sequence coding for VP2 (SEQ ID NO: 10) and a murine cytomegalovirus (IE) promoter and enhancer sequence (SEQ ID NO: 11). Positive colonies were selected based on their ability to grow in the presence of 2-deoxy-galactose (Warming Set al., Nucleic Acids Res. 2005; 33:e36) and the integration of the VP2 expression cassette was confirmed by specific PCR and sequencing.

### Cell Culture and Virus Propagation

Chicken embryonic fibroblasts (CEF) were prepared from 9-11 day old embryonated eggs of specific-pathogen-free (SPF) Rhode Island Red (RIR) birds in E199 media (Sigma) with 5% serum. DF-1 cells were propagated in Dulbecco's modified Eagles medium (DMEM, Sigma) with 10% serum. DT40 cells were propagated in RPMI-1640 medium with 10% serum. All of the cell culture media were supplemented with 100 U/ml penicillin, 100 μg/ml streptomycin and 0.25 μg/ml fungizone.

For the preparation of recombinant virus stocks, CEF were transfected with the BAC DNA from the recombinant constructs using Lipofectamine® transfection reagent (ThermoFisher) and reconstituted viruses were passaged to generate working virus stocks. Titration of SB-1 vaccine viruses was performed in CEF and the titers calculated by counting the plaque numbers four days post-infection. Recombinant virus plaques were confirmed using immunohistochemistry with IBDV VP2-specific mouse monoclonal antibody HH7 and SB-1-specific mouse monoclonal antibody Y5 (Lee LF, Liu X, Witter RL. Monoclonal antibodies with specificity for three different serotypes of Marek's disease viruses in chickens. J Immunol. 1983; 130: 1003-6) and goat anti-mouse HRP conjugated antibody (DAKO) and TrueBlue™ (KPL) peroxidase substrate.

Virulent IBDV UK661 strain for challenge was used as bursal tissue lysates from infected birds harvested at 3 days post infection (Eterradossi N et al., Zentralbl Veterinarmed B. 1992; 39: 683-91). In brief, IBDV-infected bursae were sampled from birds showing acute signs of the disease, and were homogenized in chlorotrifluoromethane (FREON 13). The supernatant collected after centrifugation at 1500 g for 30 min at 4° C. was filtered through a 0.45 um filter, and treated with penicillin and streptomycin. The lysates were titrated, aliquoted and kept at −80° C. until use. It was then inoculated intranasally to 20 four week old spf white leghorn chicken (0.05 ml per bird). D78 strain was propagated in DF-1 cells and stored at −80° C. until use. Titrations of UK661 and D78 virus strains were performed in DT40 and DF-1 cells respectively by calculating the median tissue culture infectious dose (TCID50) by the Spearman-Karber method (Brownie C et al., Biologicals. 2011; 39: 224-30).

### Virus Growth Curve Studies

Confluent CEF in 10 cm² dishes were infected in triplicate with 10³ pfu of SB-1 viruses. Following the infection, infected CEF cells were harvested at time points 0, 6, 24, 48, 96 and 120 hours post infection. The harvested cells were washed with PBS and kept at −20° C. until DNA extraction was performed.

Genomic DNA was extracted from the infected cells using QIAamp 96 DNA kit (Qiagen). Quantitation of the copy numbers for the SB-1 genome was carried out using a real-time PCR (Singh SM et al., Res Vet Sci. 2010; 89: 140-5; Islam A et al., J Virol Methods. 2004; 119: 103-13; Renz KGet al., J Virol Methods. 2006; 135: 186-91) using the primers and probes as indicated in Table 3.

TABLE 3
Real-time PCR primers

| Primers and | | |
|---|---|---|
| probes for | | |
| MDV-2 and Chicken | | SEQ |
| ovotransferrin | | IS |
| detection | Sequence | NO: |
| MDV-2 forward | AGCATGCGGGAAGAAAAGAG | 25 |
| primer | | |
| MDV-2 reverse | GAAAGGTTTTCCGCTCCCATA | 26 |
| primer | | |
| MDV-2 probe | CGCCCGTAATGCACCCGTGACT | 27 |
| Ovo forward | CACTGCCACTGGGCTCTGT | 28 |
| primer | | |
| Ovo reverse | GCAATGGCAATAAACCTCCAA | 29 |
| primer | | |
| Ovo probe | AGTCTGGAGAAGTCTGTGCAGC | 30 |
| | AGCCTCCA | |

### Serum Neutralization Test

Serum samples collected by centrifugation were heat treated at 56° C. for 30 minutes to inactivate complement factors, prior to the neutralization test. Briefly, serial dilutions of sera samples were incubated with 100 TCID50 of D78 strain of IBDV for one hour at 37° C., and serum-virus mixtures were incubated on DF-1 cell monolayers in 96 well plates for one hour, before replacing with DMEM media containing 2% FCS. The cells were checked after four days for evidence of cytopathic effects (CPE) to determine the titers.

### Statistical Analysis

For comparing the replication level of SB-1 recombinant viruses a linear model was employed. Log10 pfu was considered as the response variable and recombinant viruses in addition to hours post infection were considered as explanatory variables. Significant differences between time points and virus were identified using post-hoc Tukey tests. Differences in levels of neutralizing antibody during the course of study and between the groups were analyzed using two-way ANOVA test. The level of antibodies within each group was analyzed using one-way ANOVA test. The survival rate between groups of the birds after the challenge was compared using the Mantel-Cox test.

### Example 1

Analysis of different loci to insert an expression cassette

The construction of a bacterial artificial chromosome (BAC) comprising the SB-1 genome has been reported by Petherbridge Let al. (J Virol Methods. 2009; 158: 11-7; Singh S M et al., Res Vet Sci. 2010; 89: 140-5). Using this pSB-1 BAC clone, we examine the potential of SB-1 as a novel recombinant vector for expressing protective antigens from other avian pathogens using the well-established recombineering techniques as described above. In order to identify locations in the SB-1 genome to insert expression cassettes coding for an antigen of an avian pathogen UL10/UL11, UL3/UL4, UL21/UL22, UL40/41, UL26/27 and UL50/UL51 intergenic regions were tested to insert galk bacterial expression cassette to produce the intermediate BAC constructs. Among the listed locations, UL26/27 and UL50/51 intergenic regions produced very little amount of virus (Table 4). Further, rSB-1 UL40/41 galK produced infectious virus (Table 4), but rSB-1 UL40/41 IBDV VP2 failed to produce infectious virus (data not shown), probably due to the larger insert. As a result, these locations were excluded from the study.

TABLE 4
Comparison between different recombinants of
rSB-1 virus. The recombinant viruses were made
by inserting the indicated expression
cassette in the specified locations.

| | Titer |
|---|---|
| Name of the recombinant | (PFU/ml) |
| rSB-1 UL40/41 galK | 4 × 10⁴ |
| rSB-1 UL21/22 galK | 9.5 × 10⁴ |
| rSB-1 UL3/4 galK | 7 × 10⁴ |
| rSB-1 UL10/11 galK | 1.25 × 10⁴ |
| rSB-1 UL26/27 galK | 50 |
| rSB-1 UL50/51 galK | 1.95 × 10² |

Since integration sites UL40/41, UL26/27 and UK50/51 compromised replication we continued to further investigate insertion sites UL3/4, UL10/11 and UL21/22.

### Example 2

### Replication of rSB-1-UL3/4VP2, rSB-1-UL10/11VP2 and rSB-1-UL21/22VP2 Viruses

In this study, we used the MDV-2 (GaHV3) strain SB-1 as a novel viral vector to generate three independent constructs that expressed IBDV VP2 in the intergenic loci of UL3/4, UL10/11 or UL21/22 loci of the viral genome.

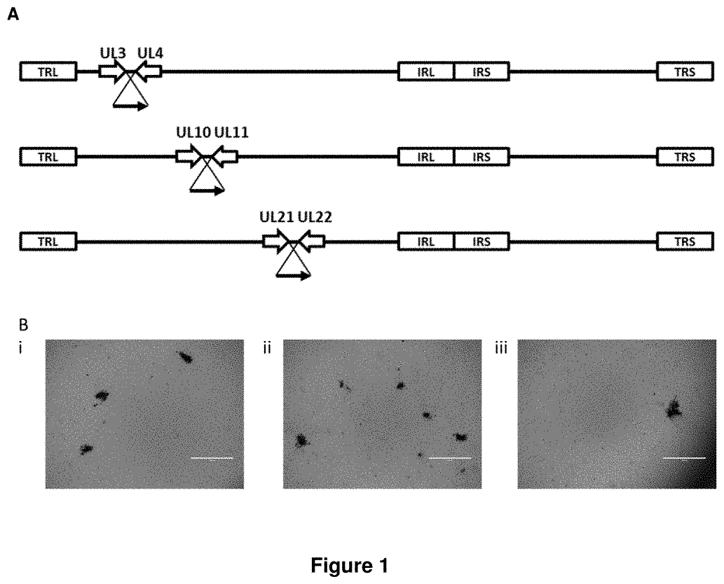

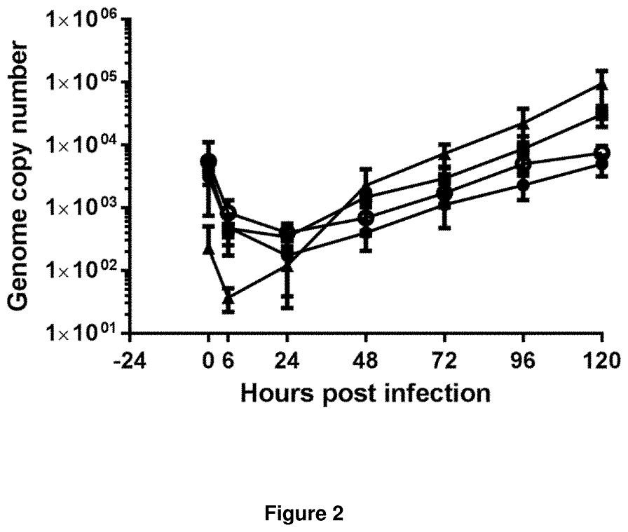

Details of the construction of pSB-1-UL3/4VP2, pSB-1-UL10/11VP2 and pSB-1-UL21/22VP2 are summarized in FIG. 1A. Recombinant SB-1 viruses produced after transfection of BAC DNA were passaged three times in CEF to produce low-passaged virus stocks. Following the rescue of the viruses, growth curve experiment was carried out to compare the replication of the recombinant and parental SB-1 viruses in CEF cells as shown in FIG. 2. No significant differences were observed in the growth rate of pSB-1 and rSB-1-UL3/4VP2 viruses between 48 and 120 hours post infection. In contrast expression of the VP2 expression cassette from the UL10/11 locus and the UL21/22 locus appeared to slow the growth of SB-1 in vitro. Such a negative effect on replication (based on titer of the virus) was also observed when inserting the galK expression cassette for rSB-1-UL10/11galK virus, but not for rSB-1-UL21/22galK virus (see Table 4). No significant difference on replication between insertion into the intergenic locus UL3/4 and pSB-1 BAC clone was observed (FIG. 2).

Therefore, it was concluded that the insertion of the VP2 expression cassette in the intragenic junction between UL3 and UL4 has the least effect on the growth rate of the virus in vitro. Expression of VP2 antigen in cells infected with rSB-1-UL3/4VP2, rSB-1-UL10/11VP2 and rSB-1-UL21/22VP2 viruses was further assessed by staining the infected cells with HH7 monoclonal and anti-mouse-HRP antibodies as shown in FIG. 1B.

### Example 3

Having narrowed down the integration sites to UL3/4, UL10/11 and UL21/22 we further examined the immunogenic potential of the VP2 protein (Fahey K J et al., J Gen Virol. 1989; 70 (Pt 6):1473-81) of infectious bursal disease virus (IBDV), the causative agent of infectious bursal disease (IBD, Gumboro disease) delivered by the recombinant SB-1 vector.

### Vaccine Development Against IBDV: Immunization Study

One-day-old SPF RIR chicks reared at the Experimental Animal House at Pirbright were used for the validation experiments. All procedures were performed in accordance with the UK Animal (Scientific Procedures) Act 1986 under Home Office Personal and Project licenses, after the approval of the internal ethical review committee.

Forty 1-day old chicks were divided into 4 groups of 10 birds each. Each of the three groups received subcutaneous injections of rSB-1-UL10/11VP2, rSB-1-UL21/22VP2 or rSB-1-UL3/4VP2 vaccine viruses respectively, each comprising 3×10³ pfu in 100 μl inoculum. Each of the 10 birds in the control group were vaccinated with 3×10³ pfu of the VAXXITEK_{HVT+IBD}® vaccine (Merial) as recommended by the manufacturer. Blood samples were collected weekly from the 2^{nd} to the 5^{th} week post-vaccination for serological studies.

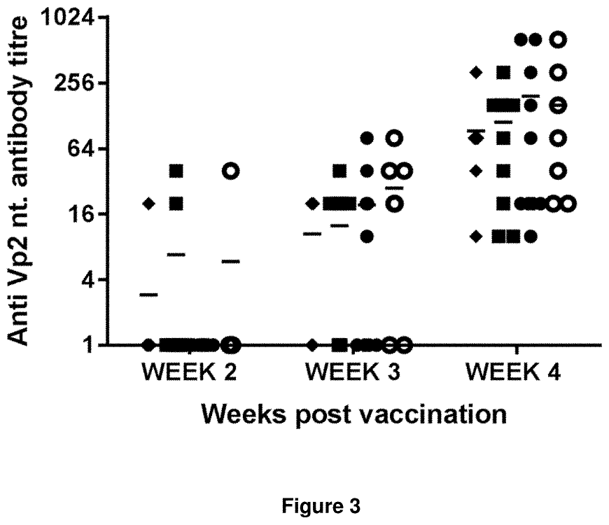

Immunogenicity of the recombinant SB-1 vaccines was assessed by measuring the production of neutralizing antibodies in vaccinated chickens. The results of vaccinating subcutaneously with VAXXITEK_{HVT+IBD}, rSB-1-UL10/11VP2, rSB-1-UL21/22VP2 or rSB-1-UL3/4VP2 vaccine virus are described in FIG. 3. Neutralizing antibodies started to appear from week two and rose to a maximum titer of 1:640 in week four post-vaccination. Neutralizing antibodies were detectable from week two onwards in all of the groups except for the group of birds vaccinated with rSB-1-UL21/22VP2, in which the level of neutralizing antibodies remained undetectable during week two. On week three post-vaccination all of the four groups showed neutralizing antibodies in the sera of about 50% of the birds. At week four post-vaccination, all of the birds showed neutralizing antibodies (FIG. 3). Although no significant differences between the mean levels of antibody between the groups were observed at each time point, the mean values of neutralizing antibodies in the groups inoculated with the experimental vaccines were higher than those of the group that received commercial vaccine.

### Example 4

For the challenge study vaccination was performed using rSB-1-UL3/4VP2 and rSB-1-UL21/22VP2. rSB-1-UL10/11VP2 was excluded from the study because of its lower growth level compared to SB-1-UL3/4VP2 and SB-1-UL21/22VP2.

### Vaccine Development Against IBDV: Challenge Study

One-day-old SPF RIR chicks reared at the Experimental Animal House at Pirbright were used for the validation experiments. All procedures were performed in accordance with the UK Animal (Scientific Procedures) Act 1986 under Home Office Personal and Project licenses, after the approval of the internal ethical review committee.

Two groups (eight birds per group) of one-day old birds were inoculated subcutaneously with 1000 pfu of rSB-1-UL3/4VP2 or rSB-1-UL21/22VP2 virus stocks. Two control groups were inoculated with 1000 pfu of the pSB-1-derived virus or with 3000 pfu of the commercial VAXXITEK_{HVT+IBD}® vaccine, respectively. After collecting the blood samples at four week post-vaccination, birds were challenged intra-nasally with 104.3 TCID50 of the virulent UK661 strain of IBDV (in a total volume of 100 μl divided between the two nostrils). In addition to the recording of the body weight, birds were monitored regularly and clinical signs scored at 6-hour intervals. Birds showing advanced clinical signs (exceeding a score of 9) were euthanized by cervical dislocation.

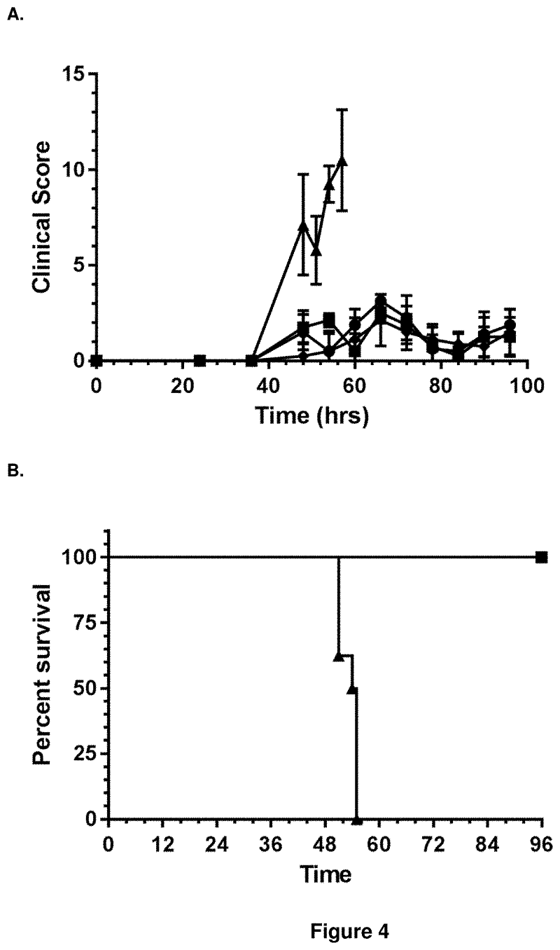

At week three post vaccination, the level of antibody in vaccinated birds was tested. One bird from the groups vaccinated with VAXXITEK_{HVT+IBD} and rSB-1-UL3/4VP2 and three birds from group vaccinated with rSB-1-UL21/22VP2 showed neutralizing antibodies in their sera. Compared to Example 3, the amount of SB-1 vaccines administered was reduced from 3000 pfu to 1000 pfu (compared to the 3000 pfu dose of VAXXITEK_{HVT+IBD} given in both experiments). Only a very limited number of birds showed neutralizing antibodies at week three post vaccination, presumably due to the smaller dose given. However, after experimental challenge with virulent virus, all of the groups (except for the SB-1 control group) showed 100% protection against IBDV. Thus our studies showed that the novel recombinant Gallid herpesvirus 2 vector vaccine induced similar levels of protection achieved from birds vaccinated with VAXXITEK_{HVT+IBD}. The mean clinical score and survival of the birds during challenge studies are shown in FIG. 4. Clinical signs were only seen in the SB-1 vaccinated control group, in which they started appearing from 36 hours post challenge and increased sharply until 56 hours post challenge when the last remaining bird was euthanized. All of the birds in the negative control group were euthanized or died 56 hours post IBDV infection, whereas all of the birds vaccinated with VAXXITEK_{HVT+IBD}, rSB-1-UL3/4VP2 and rSB-1-UL21/22VP2 survived (p<0.0001).

The results show that GaHV3 can be used as a novel vector for immunization with IBDV VP2 in 1 day old chicks, resulting in complete protection against a challenge with a 100% lethal dose of IBDV. Thus, two locations in the GaHV3 genome have been identified that permit effective delivery of the VP2 cassette with no significant cost to replication of the vector: the intergenic UL3/4 locus and the intergenic UL21/22 locus. It is predicted that these loci will also support the delivery of genes from other pathogens. The results indicate that GaHV3, particularly strain SB-1, is suitable for use as a vector for IBDV vaccination of chickens, and that it can offer 100% protection at a commercially viable dose, thereby adding a new vector platform for developing recombinant vaccines against avian diseases.

### Example 5

For studying interference ranger gold one day old chicks were inoculated in 3 separate groups with SB-1-UL3/4VP2, HVT-H9HA (HVT expressing avian influenza HA protein), a combination of HVT-H9HA and SB-1-UL3/4VP2. The birds were housed separately to minimize the possibility of shedding and transferring the vaccine virus between the groups. An additional group of birds was not-inoculated and considered as the negative control. Table 5 summarizes the four groups of birds which were used in the study.

TABLE 5
Summary of experimental design to test interaction
between the SB-1 and HVT vaccine viruses.

| | Group 1 | Group 2 | Group 3 | Group 4 |
|---|---|---|---|---|
| Vaccine | SB-1 UL3/4VP2 | HVT-H9HA | SB-1 UL3/4VP2 + | Negative |
| name | | | HVT-H9HA | control |
| Target | Target dose: | Target dose: | Target dose: | N/A |
| dose | 5000 | 5000 | 2500 pfu of each | |
| | pfu/birds | pfu/bird | vaccine | |
| Number | 10 birds | 10 birds | 10 birds | 10 birds |
| of birds | | | | |

Blood samples were collected via brachial veins of the chicken in weekly basis for 6 weeks (weeks 0, 1, 3, 4, 5 and 6). For virus neutralization assay, serum samples were heat inactivated for 30 minutes at 56° C. Following the heat inactivation, 2-fold serial dilutions of the serum were made in 96 well plates and incubated with 100 TCID50 of IBDV strain D78 to neutralize the virus. The virus and serum complex was transferred on DF-1 cells and the cells were incubated for one hour. The virus-serum complex was then removed and replaced with DMEM supplemented with 2% fetal bovine serum. Appearance of cytopathic effect (CPE) was sought after 5 days post inoculation on DF-1 cells. The last dilution of serum with the absence of CPE in DF-1 cells was reported as the titer for neutralizing antibodies in serum.

FIG. 1 summarizes the virus neutralization assay results from serum of birds that were vaccinated with SB-1 and HVT vaccines. Titers of neutralizing antibodies against IBDV VP2 for birds that were vaccinated with SB-1-UL3/4VP2, SB-1-UL3/4VP2 and HVT-9HA, and negative control are shown. As it is shown in FIG. 1, all of the birds across the 3 groups (SB-1-UL3/4VP2 vaccinated group; SB-1-UL3/4VP2 and HVT-9HA combined vaccinated group; and not vaccinated group) showed high titer of maternal antibodies prior to vaccination (week 0). Levels of the antibodies started to drop for all three groups until week 5. The actual rise in neutralizing antibodies is seen after week 5 when the titer of maternal antibodies have dropped to 64 and continued to drop further on week 6. The neutralizing antibody titer increased in the SB-1-UL3/4VP2 vaccinated group and the SB-1-UL3/4VP2 and HVT-9HA combined vaccinated group to above 1:256 at week 6. This difference between the negative group and the vaccinated groups was found to be significant using Tukey test to compare data at each time point. No statistically significant difference was observed between the SB-1-UL3/4VP2 vaccinated group and the SB-1-UL3/4VP2 and HVT-9HA combined vaccinated group at week 6. Thus, the data show that SB-1 and HVT do not interfere with each other.

The invention encompasses the following items:
1. A recombinant Gallid herpesvirus 3 (GaHV3; MDV-2) vector comprising one or more heterologous polynucleotide(s) coding for and expressing at least one antigen of an avian pathogen, inserted into the intergenic loci UL3/UL4 and/or UL21/UL22 of the Gallid herpesvirus 3 vector.
2. The recombinant Gallid herpesvirus 3 vector of item 1, wherein the Gallid herpesvirus 3 vector comprises the one or more heterologous polynucleotide(s) inserted into the intergenic locus UL3/UL4 of the Gallid herpesvirus 3 vector.
3. The recombinant Gallid herpesvirus 3 vector of item 1 or 2, wherein the recombinant Gallid herpesvirus 3 (GaHV3; MDV-2) vector is a Gallid herpesvirus 3 (GaHV3; MDV-2) strain SB-1 vector.
4. The recombinant Gallid herpesvirus 3 vector of any one of items 1 to 3, wherein the at least one antigen is protective against infectious bursal disease virus (IBDV), infectious laryngotracheitis virus (ILTV), Newcastle disease virus (NDV), avian influenza virus (AIV) or avian infectious bronchitis virus (IBV).
5. The recombinant Gallid herpesvirus 3 vector of any one of items 1 to 4, wherein the at least one antigen is selected from the group consisting of

### (a) VP2, VP3, VP4 and VPX of IBDV;

(b) glycoprotein B, glycoprotein I, glycoprotein D, glycoprotein E and glycoprotein C of ILTV;
(c) Newcastle disease virus fusion protein (NDV-F) and viral hemagglutinin neuraminidase (NDV-NH) of NDV
(d) Avian influenza hemagglutinin (HA) and neuraminidase (NA), and
(e) S1 or S2 protein of IBV.
6. The recombinant Gallid herpesvirus 3 vector of item 4 wherein the at least one antigen is protective against IBDV.
7. The recombinant Gallid herpesvirus 3 vector of any one of the preceding items, wherein the at least one antigen is VP2 of IBDV.
8. The recombinant Gallid herpesvirus 3 vector of item 7, wherein
(a) the VP2 protein amino acid sequence has at least 80% sequence identity to the sequence set forth in SEQ ID NO: 12, or
(b) the VP2 protein has the amino acid sequence set forth in SEQ ID NO: 12.
9. The recombinant Gallid herpesvirus 3 vector of any one of the preceding items, wherein the Gallid herpesvirus 3 vector contains an expression cassette(s) containing the one or more heterologous polynucleotide(s).
10. The recombinant Gallid herpesvirus 3 vector of item 9, wherein the expression cassette further comprises a promoter.
11. The recombinant Gallid herpesvirus 3 vector of item 10, wherein the promoter is selected from the group consisting of immediate early cytomegalovirus (CMV) promoter, guinea pig CMV promoter, murine CMV promoter, SV40 promoter, pseudorabies virus promoters of glycoprotein X promoter, herpes simplex virus-1 alpha 4 promoter, chicken beta-actin promoter, rabbit beta-globin promoter, herpes simplex virus thymidine kinase promoter, Marek's Disease Virus promoters of glycoproteins gC, gB, gE, or gI genes and infectious laryngotracheitis virus promoters of glycoprotein gB, gE, gI, gD genes.
12. A vaccine comprising the recombinant Gallid herpesvirus 3 vector of any one of the preceding items.
13. The vaccine of item 12 further comprising a pharmaceutically acceptable excipient, carrier or adjuvant.
14. The vaccine of item 12 or 13 comprising a further Marek's disease virus (MDV) vector selected from the group consisting of Gallid herpesvirus 3 vector, naturally attenuated MDV-1 strain Rispens (CVI-988) vector and herpesvirus of turkeys (HVT) strain Fc126 vector.
15. The vaccine of item 14 wherein the further MDV vector is a recombinant MDV vector.
16. The vaccine of item 15, wherein the recombinant Marek's disease virus (MDV) vector comprises one or more heterologous polynucleotide(s) coding for and expressing at least one antigen of an avian pathogen.
17. The vaccine of items 14 to 16, wherein the recombinant Marek's disease virus vector is a second recombinant Gallid herpesvirus 3 vector, preferably a recombinant Gallid herpesvirus 3 strain SB-1 vector, in addition to the first recombinant Gallid herpesvirus 3 vector of any one of items 1 to 11.
18. The vaccine of item 17 wherein the second recombinant Gallid herpesvirus 3 vector comprises the one or more heterologous polynucleotide(s) inserted into the intergenic loci UL3/UL4 and/or UL21/UL22 of said second Gallid herpesvirus 3 vector.
19. The vaccine of item 18, wherein the second recombinant Gallid herpesvirus 3 vector comprises the one or more heterologous polynucleotide(s) inserted into the intergenic locus UL3/UL4 of the second Gallid herpesvirus 3.
20. The vaccine of any one of items 17 to 19, wherein the second recombinant Gallid herpesvirus 3 vector comprises at least one heterologous polynucleotide coding for and expressing a different antigen of an avian pathogen as the one or more heterologous polynucleotides of the first recombinant Gallid herpesvirus 3 vector.
21. The vaccine of any one of items 14 to 20, wherein the further Marek's disease virus vector of any one of items 14 to 20 and the (first) recombinant Gallid herpesvirus 3 vector of item 12 or 13 are administered together or separate from each other.
22. An isolated DNA encoding the recombinant Gallid herpesvirus 3 vector of any one of items 1 to 11.
23. A bacterial artificial chromosome (BAC) comprising a polynucleotide coding for the recombinant Gallid herpesvirus 3 vector of any one of items 1 to 11.
24. The vaccine of any one of items 12 to 21 for use in vaccinating an avian species against one or more diseases caused by one or more avian pathogens.
25. The vaccine of any one of items 12 to 21 for use in protecting an avian species against clinical symptoms caused by one or more avian pathogens.
26. The vaccine of any one of items 12 to 21 for use in vaccinating an avian species against Marek's disease and one or more diseases caused by one or more avian pathogens.
27. The vaccine of any one of items 12 to 21 for use in protecting an avian species against clinical symptoms caused by Marek's disease virus and clinical symptoms caused by one or more avian pathogens.
28. The vaccine for use as in item 24 or 26 wherein the one or more diseases is caused by one or more of infectious bursal disease virus (IBDV), infectious laryngotracheitis virus (ILTV), Newcastle disease virus (NDV), avian influenza virus (AIV) or avian infectious bronchitis virus (IBV).
29. The vaccine for use as in any one of items 24 to 28, wherein the avian species is poultry, preferably chicken, duck, goose, turkey, quail, guinea or pigeon.
30. The vaccine for use as in item 29, wherein the avian species is turkey or chicken, preferably chicken.
31. The vaccine for use as in any one of items 24 to 30, wherein the one or more avian pathogens causing the diseases or clinical symptoms is selected from the group consisting of Newcastle disease virus, infectious bursal disease virus and avian infectious laryngotracheitis virus, avian influenza virus and avian infectious bronchitis virus (IBV).
32. The vaccine for use as in any one of items 24 to 31 wherein the vaccine is to be administered by spray administration, in ovo, subcutaneously, intramuscularly, orally or nasally.
33. The vaccine of item 32 wherein the vaccine is to be administration in ovo, preferably in ovo in 18 day old embryonated eggs.
34. The vaccine for use as in item 32, wherein the vaccine is to be administered intramuscularly or subcutaneously in chicks, preferably in 1 day old chicks.
35. A recombinant Gallid herpesvirus 3 (GaHV3; MDV-2) vector of any one of items 5 to 8 for protecting an avian species against clinical symptoms caused by infectious bursal disease virus.
36. A method of treating an avian species for protection against Marek's Disease and one or more diseases caused by one or more avian pathogens comprising the step of administering an effective amount of the vaccine of any one of items 12 to 21.
37. The method of item 36 wherein the one or more diseases is caused by one or more of infectious bursal disease virus (IBDV), infectious laryngotracheitis virus (ILTV), Newcastle disease virus (NDV), avian influenza virus (AIV) or avian infectious bronchitis virus (IBV).
38. The method of item 36 wherein the route of administration is spray administration, in ovo administration, subcutaneous administration, intramuscular administration, oral administration or nasal administration.
39. The method of item 38 wherein the route of administration is in ovo administration.
40. The method of item 36 wherein the avian species is selected from the group consisting of chicken, duck, goose, turkey, quail, guinea or pigeon.
41. The method of item 40 wherein the avian species is chicken.
42. A recombinant Gallid herpesvirus 3 (GaHV3; MDV-2) vector comprising one or more marker(s) inserted into the intergenic loci UL3/UL4 and/or UL21/UL22 of the Gallid herpesvirus 3 vector.
43. The recombinant Gallid herpesvirus 3 vector of item 42, wherein the Gallid herpesvirus 3 vector comprises the one or more marker(s) inserted into the intergenic locus UL3/UL4 of the Gallid herpesvirus 3 vector.
44. The recombinant Gallid herpesvirus 3 vector of item 42 or 43, wherein the marker is a selection marker gene, a reporter gene or a DNA bar code.
45. The recombinant Gallid herpesvirus 3 vector of item 42 or 43, wherein the Gallid herpesvirus 3 vector comprises one or more expression cassette(s) comprising a selection marker gene or a reporter gene.
46. The recombinant Gallid herpesvirus 3 vector of item 45, wherein the selection marker gene or reporter gene is galK or a kan/sacB combination.
47. A bacterial artificial chromosome (BAC) comprising a polynucleotide coding for the recombinant Gallid herpesvirus 3 vector of any one of items 42 to 46.
48. A method of producing a recombinant Gallid herpesvirus 3 vector comprising
(a) providing a Gallid herpesvirus 3 vector,
(b) inserting one or more heterologous polynucleotide(s) coding for and expressing at least one antigen of an avian pathogen into the intergenic loci UL3/UL4 and/or UL21/UL22 of the Gallid herpesvirus 3 vector, and optionally
(c) amplifying the Gallid herpesvirus 3 vector comprising one or more heterologous polynucleotide(s) coding for at least one antigen of an avian pathogen of step (b).
49. A method of producing a recombinant Gallid herpesvirus 3 vector comprising
(a) providing a Gallid herpesvirus 3 vector comprising one or more marker(s) in the intergenic loci UL3/UL4 and/or UL21/UL22 of the Gallid herpesvirus 3 vector,
(b) replacing the one or more marker(s) with an expression cassette comprising one or more heterologous polynucleotides coding for at least one antigen of an avian pathogen, and
(c) amplifying the Gallid herpesvirus 3 vector comprising one or more heterologous polynucleotide(s) coding for at least one antigen of an avian pathogen of step (b).
50. The method of item 48 or 49, wherein step (a) comprises providing a bacterial artificial chromosome comprising a polynucleotide coding for the recombinant Gallid herpesvirus 3 vector.
51. The method of item 50, wherein steps (b) and (c) are performed in E. coli.
52. The method of item 51, further comprising the following steps:
(d) isolating the bacterial artificial chromosome comprising a polynucleotide coding for the recombinant Gallid herpesvirus 3 vector comprising one or more heterologous polynucleotide(s) coding for at least one antigen of an avian pathogen of step (b)
(e) transfecting chicken embryonic fibroblasts with the bacterial artificial chromosome of step (d).
53. The method of any one of items 48 to 52, wherein the Gallid herpesvirus 3 vector is a Gallid herpesvirus 3 strain SB-1 vector.

SEQUENCE TABLE:

SEQ ID NO: 1_intergenic locus UL3/4
SEQ ID NO: 2_integenic locus UL10/11
SEQ ID NO: 3_intergenic locus UL21/22
SEQ ID NO: 4_intergenic locus UL40/41
SEQ ID NO: 5_intergenic locus UL50/51
SEQ ID NO: 6_intergenic locus UL26/27
SEQ ID NO: 7_intergenic locus UL45/46
SEQ ID NO: 8_galK expression cassette
SEQ ID NO: 9_IBDV VP2 expression cassette nucleotide sequence
SEQ ID NO: 10_IBDV VP2 nucleotide sequence
SEQ ID NO: 11_murine cytomegalovirus (IE) promoter and enhancer sequence
SEQ ID NO: 12_IBDV VP2 protein sequence
SEQ ID NO: 13_UL21/22F-GalKF primer
SEQ ID NO: 14_UL21/22R-GalKR primer
SEQ ID NO: 15_UL26/27F-GalKF primer
SEQ ID NO: 16_UL26/27R-GalKR primer
SEQ ID NO: 17_UL3.5/4F-GalKF primer
SEQ ID NO: 18_UL3.5/4R-GalKR primer
SEQ ID NO: 19_UL10/11F-GalKF primer
SEQ ID NO: 20_UL10/11R-GalKR primer
SEQ ID NO: 21_UL40/41F-GalKF primer
SEQ ID NO: 22_UL40/41R-GalKR primer
SEQ ID NO: 23_UL50/51F-GalKF primer
SEQ ID NO: 24_UL50/51R-GalKR primer
SEQ ID NO: 25_MDV-2 forward primer
SEQ ID NO: 26_MDV-2 reverse primer
SEQ ID NO: 27_MDV-2 probe
SEQ ID NO: 28_ovo forward primer
SEQ ID NO: 29_ovo reverse primer
SEQ ID NO: 30_ovo probe
SEQ ID NO: 31_Gallid herpesvirus 1 glycoprotein E (gE)
SEQ ID NO: 32_Gallid herpesvirus 1 glycoprotein I (gI)
SEQ ID NO: 33_Gallid herpesvirus 1 glycoprotein B (gB)
SEQ ID NO: 34_Newcastle disease virus fusion protein
SEQ ID NO: 35_Newcastle disease virus hemagglutinin-neuraminidase (HN)
SEQ ID NO: 36_Influenza virus A serotype H5 hemagglutinin (H5 HA)
SEQ ID NO: 37_Influenza virus A serotype H7 hemagglutinin (H7 HA)
SEQ ID NO: 38_Influenza virus A serotype H9 hemagglutinin (H9 HA)
SEQ ID NO: 39_Influenza virus A serotype H5N1 neuraminidase (NA)
SEQ ID NO: 40_Infectious bronchitis virus, spike glycoprotein

1. A recombinant Gallid herpesvirus 3 (GaHV3; MDV-2) vector comprising one or more heterologous polynucleotides coding for and expressing at least one antigen of an avian pathogen, wherein the one or more heterologous polynucleotides are inserted into intergenic locus UL21/UL22 of the Gallid herpesvirus 3 vector.

2. The recombinant Gallid herpesvirus 3 vector of claim 1, wherein the recombinant Gallid herpesvirus 3 (GaHV3; MDV-2) vector is a Gallid herpesvirus 3 (GaHV3; MDV-2) strain SB-1 vector.

3. The recombinant Gallid herpesvirus 3 vector of claim 1, wherein the at least one antigen is protective against infectious bursal disease virus (IBDV), infectious laryngotracheitis virus (ILTV), Newcastle disease virus (NDV), avian influenza virus (AIV) and/or avian infectious bronchitis virus (IBV).

4. A vaccine comprising the recombinant Gallid herpesvirus 3 vector of claim 1, optionally further comprising a pharmaceutically acceptable excipient, carrier or adjuvant.

5. The vaccine of claim 4 comprising a further Marek's disease virus (MDV) vector selected from the group consisting of Gallid herpesvirus 3 vector, naturally attenuated MDV-1 strain Rispens (CVI-988) vector and herpesvirus of turkeys (HVT) strain Fc126 vector.

6. The vaccine of claim 5, wherein the further MDV vector is a recombinant MDV vector and the recombinant MDV vector comprises one or more heterologous polynucleotides coding for and expressing at least one antigen of an avian pathogen.

7. The vaccine of claim 6, wherein the recombinant MDV vector is a second recombinant Gallid herpesvirus 3 vector, and wherein
(a) the second recombinant Gallid herpesvirus 3 vector comprises one or more heterologous polynucleotides inserted into intergenic locus UL21/UL22 of said second Gallid herpesvirus 3 vector; and
(b) the second recombinant Gallid herpesvirus 3 vector comprises at least one heterologous polynucleotide coding for and expressing a different antigen of an avian pathogen as the one or more heterologous polynucleotides of the first recombinant Gallid herpesvirus 3 vector.

8. A bacterial artificial chromosome (BAC) comprising a polynucleotide coding for the recombinant Gallid herpesvirus 3 vector of claim 1.

9. A method of vaccinating an avian against Marek's disease or one or more diseases caused by one or more avian pathogens, comprising administering the vaccine of claim 4 to the avian.

10. A method of protecting an avian against clinical symptoms caused by Marek's disease virus or clinical symptoms caused by one or more avian pathogens, comprising administering the vaccine of claim 4 to the avian.

11. The method of claim 9, wherein
the one or more diseases are caused by one or more of infectious bursal disease virus (IBDV), infectious laryngotracheitis virus (ILTV), Newcastle disease virus (NDV), avian influenza virus (AIV) or avian infectious bronchitis virus (IBV).

12. A recombinant Gallid herpesvirus 3 (GaHV3; MDV-2) vector comprising one or more markers inserted into intergenic locus UL21/UL22 of the Gallid herpesvirus 3 vector, wherein the one or more markers comprise a selection marker gene, a reporter gene or a DNA bar code.

13. A method of producing a recombinant Gallid herpesvirus 3 vector according to claim 1, comprising
(a) providing a Gallid herpesvirus 3 vector,
(b) inserting one or more heterologous polynucleotides coding for and expressing at least one antigen of an avian pathogen into intergenic locus UL21/UL22 of the Gallid herpesvirus 3 vector, and optionally
(c) amplifying the Gallid herpesvirus 3 vector comprising one or more heterologous polynucleotides coding for at least one antigen of an avian pathogen of step (b).

14. A method of producing the recombinant Gallid herpesvirus 3 vector of claim 1, comprising
(a) providing a Gallid herpesvirus 3 vector comprising one or more markers in intergenic locus UL21/UL22 of the Gallid herpesvirus 3 vector,
(b) replacing the one or more markers with an expression cassette comprising one or more heterologous polynucleotides coding for at least one antigen of an avian pathogen, and
(c) amplifying the Gallid herpesvirus 3 vector comprising one or more heterologous polynucleotides coding for at least one antigen of an avian pathogen of step (b).

15. The method of claim 13, wherein step (a) comprises providing a bacterial artificial chromosome comprising a polynucleotide coding for the recombinant Gallid herpesvirus 3 vector and wherein the method optionally further comprises the steps of
(d) isolating the bacterial artificial chromosome comprising a polynucleotide coding for the recombinant Gallid herpesvirus 3 vector comprising one or more heterologous polynucleotides coding for at least one antigen of an avian pathogen of step (b); and
(e) transfecting chicken embryonic fibroblasts with the bacterial artificial chromosome of step (d).

16. The Gallid herpesvirus 3 vector of claim 3, wherein the at least one antigen is selected from the group consisting of:
(a) VP2, VP3, VP4, and VPX of IBDV;
(b) glycoprotein B, glycoprotein I, glycoprotein D, glycoprotein E, and glycoprotein C of ILTV;
(c) Newcastle disease virus fusion protein (NDV-F) and viral hemagglutinin neuraminidase (NDV-NH) of NDV;
(d) Avian influenza hemagglutinin (HA) and neuraminidase (NA); and
(e) S1 or S2 protein of IBV.

17. The vaccine of claim 7, wherein the recombinant MDV vector is a recombinant Gallid herpesvirus 3 strain SB-1 vector.

18. The method of claim 9, wherein the avian is a duck, goose, chicken, turkey, guinea, quail, or pigeon.

19. The method of claim 10, wherein the one or more clinical symptoms are caused by one or more of infectious bursal disease virus (IBDV), infectious laryngotracheitis virus (ILTV), Newcastle disease virus (NDV), avian influenza virus (AIV) or avian infectious bronchitis virus (IBV).

20. The method of claim 19, wherein the avian is a duck, goose, chicken, turkey, guinea, quail, or pigeon.


SEQUENCE LISTING (40 sequences)

SEQ ID NO: 1 - DNA, 96 bp, Gallid herpesvirus 3
  intergenic locus UL3/4
caagaagcatctaaaacgcgcttgattgtcgagtggctgaataaaatctttattgatcgactcgctttcctatttctgatttaataaccatagatg

SEQ ID NO: 2 - DNA, 100 bp, Gallid herpesvirus 3
  intergenic locus UL21/22
gccgggcatatacagaacgtaagccaagctggagtttgtgtaagtatgtgctctacagcgtgcgggaagggcggttcgcgaataaacacaacagtacagg

SEQ ID NO: 3 - DNA, 100 bp, Gallid herpesvirus 3
  intergenic locus UL10/11
agtgaatgggatgaataggaacgcccgaaacataataaaacgctaaatctacaagtgattgtcgcgcgacttatttatcactataacgtatcgttacatt

SEQ ID NO: 4 - DNA, 100 bp, Gallid herpesvirus 3
  intergenic locus UL40/41
gtatcgaacgatctttaattagcctgtgtgcaactgtactttctacccctacccacaagaattaataaatgaattcaaattatcgcttttcgcaccgtgt

SEQ ID NO: 5 - DNA, 101 bp, Gallid herpesvirus 3
  intergenic locus UL50/51
aaacggcagcattcgatacggaatcgggcaggagcgagagagagtgtgcgtatgtaatcgtgcgcaactatacattattgcccgctcgacccgaagccgtc

SEQ ID NO: 6 - DNA, 100 bp, Gallid herpesvirus 3
  intergenic locus UL26/27
ggatccgctatgtcgacgtataagtttatacattttgcgaccgcaatagcaaataaaagtaaaataatcgtatgcgcacgtgagaatttatttatcgaga

SEQ ID NO: 7 - DNA, 100 bp, Gallid herpesvirus 3
  intergenic locus UL45/46
gagagaccgagcattagagtagcacttatttattctatcgcagagaaacagcgcgcgttcaaaaaaaacacaggcggggtacgataaatttacgcggccg

SEQ ID NO: 8 - DNA, 1236 bp, Artificial Sequence
  galK expression cassette
gaattcctgttgacaattaatcatcggcatagtatatcggcatagtataatacgacaaggtgaggaactaaacccaggaggcagatcatgagtctgaaagaaaaaacacaatctctgtttgccaacgcatttggctaccctgccactcacaccattcaggcgcctggccgcgtgaatttgattggtgaacacaccgactacaacgacggtttcgttctgccctgcgcgattgattatcaaaccgtgatcagttgtgcaccacgcgatgaccgtaaagttcgcgtgatggcagccgattatgaaaatcagctcgacgagttttccctcgatgcgcccattgtcgcacatgaaaactatcaatgggctaactacgttcgtggcgtggtgaaacatctgcaactgcgtaacaacagcttcggcggcgtggacatggtgatcagcggcaatgtgccgcagggtgccgggttaagttcttccgcttcactggaagtcgcggtcggaaccgtattgcagcagctttatcatctgccgctggacggcgcacaaatcgcgcttaacggtcaggaagcagaaaaccagtttgtaggctgtaactgcgggatcatggatcagctaatttccgcgctcggcaagaaagatcatgccttgctgatcgattgccgctcactggggaccaaagcagtttccatgcccaaaggtgtggctgtcgtcatcatcaacagtaacttcaaacgtaccctggttggcagcgaatacaacacccgtcgtgaacagtgcgaaaccggtgcgcgtttcttccagcagccagccctgcgtgatgtcaccattgaagagttcaacgctgttgcgcatgaactggacccgatcgtggcaaaacgcgtgcgtcatatactgactgaaaacgcccgcaccgttgaagctgccagcgcgctggagcaaggcgacctgaaacgtatgggcgagttgatggcggagtctcatgcctctatgcgcgatgatttcgaaatcaccgtgccgcaaattgacactctggtagaaatcgtcaaagctgtgattggcgacaaaggtggcgtacgcatgaccggcggcggatttggcggctgtatcgtcgcgctgatcccggaagagctggtgcctgccgtacagcaagctgtcgctgaacaatatgaagcaaaaacaggtattaaagagactttttacgtttgtaaaccatcacaaggagcaggacagtgctga

SEQ ID NO: 9 - DNA, 3031 bp, Artificial Sequence
  IBDV VP2 expression cassette
ggatcccccaactccgcccgttttatgactagaaccaatagtttttaatgccaaatgcactgaaatcccctaatttgcaaagccaaacgccccctatgtgagtaatacggggactttttacccaatttcccaagcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagttccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcaggcggccgctctagaactcgtcgatcgcagcgatgataaacctgcaagatcaaacccaacagattgttccgttcatacggagccttctgatgccaacaaccggaccggcgtccattccggacgacaccctggagaagcacactctcaggtcagagacctcgacctacaatttgactgtgggggacacagggtcagggctaattgtctttttccctggattccctggctcaattgtgggtgctcactacacactgcagagcaatgggaactacaagttcgatcagatgctcctgactgcccagaacctaccggccagctacaactactgcagactagtgagtcggagtctcacagtgaggtcaagcacactccctggtggcgtttatgcactaaacggcaccataaacgccgtgaccttccaaggaagcctgagtgaactgacagatgttagctacaatgggttgatgtctgcaacagccaacatcaacgacaaaattgggaatgtcctggtaggggaaggggtcactgtcctcagcctacccacatcatatgatcttgggtatgtgaggcttggtgaccccattcccgctatagggcttgacccaaaaatggtagctacatgcgacagcagtgacaggcccagagtctacaccataactgcagccgatgattaccaattctcatcacagtaccaaccaggtggggtaacaatcacactgttctcagccaacattgatgctatcacaagcctcagcattgggggagagctcgtgtttcaaacaagcgtccaaggccttgtactgggcgccaccatctaccttataggctttgatgggactgcggtaatcaccagagctgtagccgcagataatgggctgacggccggcaccgacaatcttatgccattcaatcttgtcattccaaccaatgagataacccagccaatcacatccatcaaactggagatagtgacctccaaaagtggtggtcaggcaggggatcagatgtcatggtcggcaagtgggagcctagcagtgacgatccatggtggcaactatccaggggccctccgtcccgtcacactagtagcctacgaaagagtggcaacaggatccgtcgttacggtcgctggggtgagtaacttcgagctgattccaaatcctgaactagcaaagaacctggttacagaatacggccgatttgacccaggagccatgaactacacaaaattgatactgagtgagagggaccgtcttggcatcaagaccgtctggccaacaagggagtacactgattttcgtgagtacttcatggaggtggccgacctcaactctcccctgaagattgcaggagcatttggcttcaaagacataatccgggctataaggaggtaagcttgatctagagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgac

SEQ ID NO: 10 - DNA, 1362 bp, Infectious bursal disease virus 52/70
  IBDV VP2 nucleotide sequence
atgataaacctgcaagatcaaacccaacagattgttccgttcatacggagccttctgatgccaacaaccggaccggcgtccattccggacgacaccctggagaagcacactctcaggtcagagacctcgacctacaatttgactgtgggggacacagggtcagggctaattgtctttttccctggattccctggctcaattgtgggtgctcactacacactgcagagcaatgggaactacaagttcgatcagatgctcctgactgcccagaacctaccggccagctacaactactgcagactagtgagtcggagtctcacagtgaggtcaagcacactccctggtggcgtttatgcactaaacggcaccataaacgccgtgaccttccaaggaagcctgagtgaactgacagatgttagctacaatgggttgatgtctgcaacagccaacatcaacgacaaaattgggaatgtcctggtaggggaaggggtcactgtcctcagcctacccacatcatatgatcttgggtatgtgaggcttggtgaccccattcccgctatagggcttgacccaaaaatggtagctacatgcgacagcagtgacaggcccagagtctacaccataactgcagccgatgattaccaattctcatcacagtaccaaccaggtggggtaacaatcacactgttctcagccaacattgatgctatcacaagcctcagcattgggggagagctcgtgtttcaaacaagcgtccaaggccttgtactgggcgccaccatctaccttataggctttgatgggactgcggtaatcaccagagctgtagccgcagataatgggctgacggccggcaccgacaatcttatgccattcaatcttgtcattccaaccaatgagataacccagccaatcacatccatcaaactggagatagtgacctccaaaagtggtggtcaggcaggggatcagatgtcatggtcggcaagtgggagcctagcagtgacgatccatggtggcaactatccaggggccctccgtcccgtcacactagtagcctacgaaagagtggcaacaggatccgtcgttacggtcgctggggtgagtaacttcgagctgattccaaatcctgaactagcaaagaacctggttacagaatacggccgatttgacccaggagccatgaactacacaaaattgatactgagtgagagggaccgtcttggcatcaagaccgtctggccaacaagggagtacactgattttcgtgagtacttcatggaggtggccgacctcaactctcccctgaagattgcaggagcatttggcttcaaagacataatccgggctataaggaggtaa

SEQ ID NO: 11 - DNA, 1206 bp, Artificial Sequence
  murine CMV IE promoter and enhancer sequence
gttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagttccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcaggcggccgctctagaactcgtcgatcgcagcg

SEQ ID NO: 12 - protein, 453 aa, Infectious bursal disease virus 52/70
  IBVD VP protein sequence
MINLQDQTQQIVPFIRSLLMPTTGPASIPDDTLEKHTLRSETSTYNLTVGDTGSGLIVFFPGFPGSIVGAHYTLQSNGNYKFDQMLLTAQNLPASYNYCRLVSRSLTVRSSTLPGGVYALNGTINAVTFQGSLSELTDVSYNGLMSATANINDKIGNVLVGEGVTVLSLPTSYDLGYVRLGDPIPAIGLDPKMVATCDSSDRPRVYTITAADDYQFSSQYQPGGVTITLFSANIDAITSLSIGGELVFQTSVQGLVLGATIYLIGFDGTAVITRAVAADNGLTAGTDNLMPFNLVIPTNEITQPITSIKLEIVTSKSGGQAGDQMSWSASGSLAVTIHGGNYPGALRPVTLVAYERVATGSVVTVAGVSNFELIPNPELAKNLVTEYGRFDPGAMNYTKLILSERDRLGIKTVWPTREYTDFREYFMEVADLNSPLKIAGAFGFKDIIRAIRR

SEQ ID NO: 13 - DNA, 74 bp, Artificial Sequence
  forward primer UL21/22F-GalKF primer
gccgggcatatacagaacgtaagccaagctggagtttgtgtaagtatgtgcctgttgacaattaatcatcggca

SEQ ID NO: 14 - DNA, 71 bp, Artificial Sequence
  reverse primer UL21/22R-GalKR
cctgtactgttgtgtttattcgcgaaccgcccttcccgcacgctgtagagtcagcactgtcctgctccttg

SEQ ID NO: 15 - DNA, 74 bp, Artificial Sequence
  forward primer UL26/27F-GalKF
ggatccgctatgtcgacgtataagtttatacattttgcgaccgcaatagccctgttgacaattaatcatcggca

SEQ ID NO: 16 - DNA, 71 bp, Artificial Sequence
  reverse primer UL26/27R-GalKR
tctcgataaataaattctcacgtgcgcatacgattattttacttttattttcagcactgtcctgctccttg

SEQ ID NO: 17 - DNA, 74 bp, Artificial Sequence
  forward primer UL3/4F-GalKF
taaaacgcgcttgattgtcgagtggctgaataaaatctttattgatcgaccctgttgacaattaatcatcggca

SEQ ID NO: 18 - DNA, 70 bp, Artificial Sequence
  reverse primer UL3/4R-GalKR
atagattccccgccccatctatggttattaaatagaaataggaaagcgatcagcactgtcctgctccttg

SEQ ID NO: 19 - DNA, 74 bp, Artificial Sequence
  forward primer UL10/11F-GalKF
agtgaatgggatgaataggaacgcccgaaacataataaaacgctaaatctcctgttgacaattaatcatcggca

SEQ ID NO: 20 - DNA, 71 bp, Artificial Sequence
  reverse primer UL10/11R-GalKR
aatgtaacgatacgttatagtgataaataagtcgcgcgacaatcacttgttcagcactgtcctgctccttg

SEQ ID NO: 21 - DNA, 74 bp, Artificial Sequence
  forward primer UL40/41F-GalKF
gtatcgaacgatctttaattagcctgtgtgcaactgtactttctacccctcctgttgacaattaatcatcggca

SEQ ID NO: 22 - DNA, 71 bp, Artificial Sequence
  reverse primer UL40/41R-GalKR
acacggtgcgaaaagcgataatttgaattcatttattaattcttgtgggttcagcactgtcctgctccttg

SEQ ID NO: 23 - DNA, 74 bp, Artificial Sequence
  forward primer UL50/51F-GalKF
gaaacggcagcattcgatacggaatcgggcaggagcgagagagagtgtgccctgttgacaattaatcatcggca

SEQ ID NO: 24 - DNA, 71 bp, Artificial Sequence
  reverse primer UL50/51R-GalKR
gtatgtaatcgtgcgcaactatacattattgcccgctcgacccgaagccgtcagcactgtcctgctccttg

SEQ ID NO: 25 - DNA, 20 bp, Artificial Sequence
  MDV-2 Forward primer
agcatgcgggaagaaaagag

SEQ ID NO: 26 - DNA, 21 bp, Artificial Sequence
  MDV-2 Reverse primer
gaaaggttttccgctcccata

SEQ ID NO: 27 - DNA, 22 bp, Artificial Sequence
  MDV-2 PROBE
cgcccgtaatgcacccgtgact

SEQ ID NO: 28 - DNA, 19 bp, Artificial Sequence
  Ovo forward primer
cactgccactgggctctgt

SEQ ID NO: 29 - DNA, 21 bp, Artificial Sequence
  Ovo reverse primer
gcaatggcaataaacctccaa

SEQ ID NO: 30 - DNA, 30 bp, Artificial Sequence
  Ovo probe
agtctggagaagtctgtgcagcagcctcca

SEQ ID NO: 31 - protein, 499 aa, Gallid herpesvirus 1
  glycoprotein E
MNMLVIVLASCLARLTFATRHVLFLEGTQAVLGEDDPRNVPEGTVIKWTKVLRNACKMKAADVCSSPNYCFHDLIYDGGKKDCPPAGPLSANLVILLKRGESFVVLGSGLHNSNITNIMWTEYGGLLFDPVTRSDEGIYFRRISQPDLAMETTSYNVSVLSHVDEKAPAPHEVEIDTIKPSEAHAHVELQMLPFHELNDNSPTYVTPVLRVFPPTEHVKFNVTYSWYGFDVKEECEEVKLFEPCVYHPTDGKCQFPATNQRCLIGSVLMAEFLGAASLLDCSRDTLEDCHENRVPNLRFDSRLSESRAGLVISPLIAIPKVLIIVVSDGDILGWSYTVLGKRNSPRVVVETHMPSKVPMNKVVIGSPGPMDETGNYKMYFVVAGVTATCVILTCALLVGKKKCPAHQMGTFSKTEPLYAPLPKNEFEAGGLTDDEEVIYDEVYEPLFRGYCKQEFREDVNTFFGAVVEGERALNFKSAIASMADRILANKSGRRNMDSY

SEQ ID NO: 32 - protein, 362 aa, Gallid herpesvirus 1
  glycoprotein I
MASLLGTLALLAATLAPFGAMGIVITGNHVSARIDDDHIVIVAPRPEATIQLQLFFMPGQRPHKPYSGTVRVAFRSDITNQCYQELSEERFENCTHRSSSVFVGCKVTEYTFSASNRLTGPPHPFKLTIRNPRPNDSGMFYVIVRLDDTKEPIDVFAIQLSVYQFANTAATRGLYSKASCRTFGLPTVQLEAYLRTEESWRNWQAYVATEATTTSAEATTPTPVTATSASELEAEHFTFPWLENGVDHYEPTPANENSNVTVRLGTMSPTLIGVTVAAVVSATIGLVIVISIVTRNMCTPHRKLDTVSQDDEERSQTRRESRKFGPMVACEINKGADQDSELVELVAIVNPSALSSPDSIKM

SEQ ID NO: 33 - protein, 883 aa, Gallid herpesvirus 1
  glycoprotein B
MQSYIAVNIDMASLKMLICVCVAILIPSTLSQDSHGIAGIIDPRDTASMDVGKISFSEAIGSGAPKEPQIRNRIFACSSPTGASVARLAQPRHCHRHADSTNMTEGIAVVFKQNIAPYVFNVTLYYKHITTVTTWALFSRPQITNEYVTRVPIDYHEIVRIDRSGECSSKATYHKNFMFFEAYDNDEAEKKLPLVPSLLRSTVSKAFHTTNFTKRHQTLGYRTSTSVDCVVEYLQARSVYPYDYFGMATGDTVEISPFYTKNTTGPRRHSVYRDYRFLEIANYQVRDLETGQIRPPKKRNFLTDEQFTIGWDAMEEKESVCTLSKWIEVPEAVRVSYKNSYHFSLKDMTMTFSSGKQPFNISRLHLAECVPTIATEAIDGIFARKYSSTHVRSGDIEYYLGSGGFLIAFQKLMSHGLAEMYLEEAQRQNHLPRGRERRQAAGRRTASLQSGPQGDRITTHSSATFAMLQFAYDKIQAHVNELIGNLLEAWCELQNRQLIVWHEMKKLNPNSLMTSLFGQPVSARLLGDIVAVSKCIEIPIENIRMQDSMRMPGDPTMCYTRPVLIFRYSSSPESQFSANSTENHNLDILGQLGEHNEILQGRNLIEPCMINHRRYFLLGENYLLYEDYTFVRQVNASEIEEVSIFINLNATILEDLDFVPVEVYTREELRDTGTLNYDDVVRYQNIYNKRFRDIDTVIRGDRGDAIFRAIADFFGNTLGEVGKALGTVVMTAAAAVISTVSGIASFLSNPFAALGIGIAVVVSIILGLLAFKYVMNLKSNPVQVLFPGAVPPAGTPPRPSRRYYKDEEEVEEDSDEDDRILATRVLKGLELLHKDEQKARRQKARFSAFAKNMRNLFRRKPRTKEDDYPLLEYPSWAEESEDE

SEQ ID NO: 34 - protein, 553 aa, Newcastle disease virus
  NDV-F
MGSRPFTKNPAPMMLTIRVALVLSCICPANSIDGRPFAAAGIVVTGDKAVNIYTSSQTGSIIVKLLPNLPKDKEACAKAPLDAYNRTLTTLLTPLGDSIRRIQESVTTSGGGRQGRLIGAIIGGVALGVATAAQITAAAALIQAKQNAANILRLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGIGNNQLSSLIGSGLITGNPILYDSQTQLLGIQVTLPSVGNLNNMRATYLETLSVSTTRGFASALVPKVVTQVGSVIEELDTSYCIETDLDLYCTRIVTFPMSPGIYSCLSGNTSACMYSKTEGALTTPYMTIKGSVIANCKMTTCRCVNPPGIISQNYGEAVSLIDKQSCNVLSLGGITLRLSGEFDVTYQKNISIQDSQVIITGNLDISTELGNVNNSISNALNKLEESNRKLDKVNVKLTSTSALITYIVLTIISLVFGILSLILACYLMYKQKAQQKTLLWLGNNTLDQMRATTKM

SEQ ID NO: 35 - protein, 577 aa, Newcastle disease virus
  NDV-NH
MDRAVSQVALENDEREAKNTWRLIFRIAILFLTVVTLAISVASLLYSMGASTPSDLVGIPTRISRAEEKITSTLGSNQDVVDRIYKQVALESPLALLNTETTIMNAITSLSYQINGAANNSGWGAPIHDPDYIGGIGKELIVDDASDVTSFYPSAFQEHLNFIPAPTTGSGCTRIPSFDMSATHYCYTHNVILSGCRDHSHSYQYLALGVLRTSATGRVFFSTLRSINLDDTQNRKSCSVSATPLGCDMLCSKATETEEEDYNSAVPTRMVHGRLGFDGQYHEKDLDVTTLFGDWVANYPGVGGGSFIDSRVWFSVYGGLKPNSPSDTVQEGKYVIYKRYNDTCPDEQDYQIRMAKSSYKPGRFGGKRIQQAILSIKVSTSLGEDPVLTVPPNTVTLMGAEGRILTVGTSHFLYQRGSSYFSPALLYPMTVSNKTATLHSPYTFNAFTRPGSIPCQASARCPNSCVTGVYTDPYPLIFYRNHTLRGVFGTMLDGEQARLNPASAVFDSTSRSRITRVSSSSIKAAYTTSTCFKVVKTNKTYCLSIAEISNTLFGEFRIVPLLVEILKDDGVREARSG

SEQ ID NO: 36 - protein, 568 aa, Influenza A virus
  H5 HA
MEKIVLLFAIVSLVKSDHICIGYHANNSTEQVDTIMEKNVTVTHAQDILEKTHNGKLCDLNGVKPLILKDCSVAGWLLGNPKCDEFIDVPEWSYIVEKANPANDLCYPGNFNDYEELKHLLSRINHFEKIRIIPKDSWQDHEASLGVSAACSHQGNSSFFRNVVWLLKKDDAYPIIKKSYNNTNKEDLLVLWGIHHPNDGAEQIKLYQNPTTYVSIGTSTLNQRLVPRIATRSKVNGQSGRIDFFWTILKPNDAINFESNGNFIAPEYAYKIVKKGDSTIMRSEVEYGNCSTRCQTPMGAINSSMPFHNIHPLTIGECPKYVKSKKLVLATGLRNSPQIERRRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLVLAIMVAGLSLWMCSNGSLQCRICI

SEQ ID NO: 37 - protein, 560 aa, Influenza A virus
  H7 HA
MNTQILVFIACVLIKAKGDKICLGHHAVANGTKVNTLTERGIEVVNATETVETANIEKICTKGKRPTDLGQCGLLGTLIGPPQCDQFLEFESDLIIERREGNDVCYPGKFTNEESLRQILRGSGGVDKESMGFTYSGIRTNGATSACRRSGSSFYAEMKWLLSNSDNAAFPQMTKSYRNPRNKPALIVWGIHHSGSTTEQTKLYGSGNKLITVGSSKYQQSFTPSPGARPQVNGQSGRIDFHWLLLDPNDTVTFTFNGAFIAPDRASFFRGESLGVQSDVPLDSSCGGDCFHSGGTIVSSLPFQNINSRTVGKCPRYVKQSSLLLATGMRNVPENPKTRGLFGAIAGFIENGWEGLIDGWYGFRHQNAQGEGIAADYKSTQSAIDQITGKLNRLIDKTNQQFELIDNEFNEIEQQIGNVINWTRDSMTEVWSYNAELLVAMENQHTIDLADSEMNKLYERVRKQLRENAEEDGTGCFEIFHKCDDQCMESIRNNTYDHTQYRAESLQNRIQIDPVKLSSGYKDIILWFSFGASCFLLLAIAMGLVFICIKNGNMRCTICI

SEQ ID NO: 38 - protein, 560 aa, Influenza A virus
  H9 HA
METVSLITILLVATVSNADKICIGYQSTNSTETVDTLTENNVPVTHAKELLHTEHNGMLCATSLGHPLILDTCTIEGLIYGNPSCDLLLGGREWSYIVERPSAVNGLCYPGNVENLEELRSLFSSARSYQRIQIFPDTIWNVSYSGTSKACSDSFYRSMRWLTQKNNAYPIQDAQYTNNQEKNILFMWGINHPPTDTAQTNLYTRTDTTTSVATEEINRIFKPLIGPRPLVNGLMGRIDYYWSVLKPGQTLRIRSNGNLIAPWYGHILSGESHGRILKTDLKRGSCTVQCQTEKGGLNTTLPFQNVSKYAFGNCSKYIGIKSLKLAVGLRNVPSRSSRGLFGAIAGFIEGGWSGLVAGWYGFQHSNDQGVGMAADRDSTQRAIDKITSKVNNIVDKMNKQYEIIDHEFSEVETRLNMINNKIDDQIQDIWAYNAELLVLLENQKTLDEHDANVNNLYNKVKRALGSNAVEDGKGCFELYHKCDDQCMETIRNGTYNRRKYQEESKLERQKIEGVKLESEGTYKILTIYSTVASSLVIAMGFAAFLFWAMSNGSCRCNICI

SEQ ID NO: 39 - protein, 449 aa, Influenza A virus
  H5N1 neuraminidase
MNPNRKIITIGSICMVIGIVSLMLQIGNIISIWVSHSIQTENQHQSEPISNTNFLTEKAVASVTLAGNSSLCPISGWAVHSKDNGIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASYKIFKMEKGKVVKSVELDAPNYHYEECSCYPDAGEITCVCRDNWHGSNRPWVSFNQNLEYQIGYICSGVFGDNPRPNDGTGSCGPMSLNGAYGVKGFSFKYGNGVWIGRTKSTNSRSGFEMIWDPNGWTGTDSSFSVKQDIVAITDWSGYSGSFVQHPELTGLDCIRPCFWIELIRGRPKESTIWTSGSSISFCGVNSDTVGWSWPDGAELPFTIDK

SEQ ID NO: 40 - protein, 1158 aa, Infectious bronchitis virus
  spike protein
MLGKSLLIVTVLFALCSATLYTHDYVYYYQSAYRPPNGWHLQGGAYAVVNSTNKTNNAGAASECSVGVLFNYTNGNDVGYNNSASSVAMTAPLPGMSWSKTQFCTAHCNFSDFTVFVTHCFANSCPLTGRIEKNHIRISAMRNGSLFYNLTVSVSKYPKFKSLQCVNNFTSVYLNGDLVFTSNKTTDVIGAGVYFKAGGPITYKIMKEFKVLAYFVNGTVQDVILCDDTPRGLLACQYNTGNFSDGFYPFTNSSLVKKKFIVYRESSVNTTLILTNYTFYNVTNAPPNQGGVQSILTYQTQTAQSGYYNFNLSFLSSFVYKQSDYMYGSYHPACNFRLETINNGLWFNSLSVSLAYGPLQGGCKQSVFSSRATCCYAYSYNGPRACKGVYAGELRQNFECGLLVYVTKSDGSRIQTATEPPVITQHNYNNITLNTCVDYNIYGRVGRGFITNVTDLSSSYNYLADGGLAILDTSGAIDIFVVQGEHGFNYYKVNPCEDVNQQFVVSGGKLVGILTSRNASGSQPLENQFYIKLTKETRRFRRSTSENVTSCHYVSYGRFCIQPDGSIKQIVPQELQNFVAPLLNVTENVLIPNSFNLTVTDEYIQTRMDKVQINCLQYVCGNSLDCRKLFQQYGPVCDNILSIVNSVGQKEDMELLSFYSSTKPAGYNAPVFSNISTGDFNISLLLTPSSSPRGRSFIEDLLFTSVETVGLPTDAEYKKCTAGPLGTLKDLVCAREYNGLLVLPPIITADMQTMYTASLVASMAFGGITAAGAIPFATQIQARINHLGITQSLLLKNQERIAASFNKAIGHMQEGFRSTSLALQQVQDVVNKQSAILTETMNSLNKNFGAISSVIQDIYAQLDAIQADAQVDRLITGRLSSLSVLASAKQSEYIRVSQQRELATQKINECVKSQSNRYGFCGSGRHVLSIPQNAPNGIVFIHFTYTPESFVNVTAIVGFCVQPANASQYAIVPVNGRGIFIQVNGSYYITARDMYMPRDITAGDIVTLTSCQANYVNVNKTVITTFVEDDDFDFDDELSKWWNDTKHELPDFDDFNYTVPILNISGEIDRIQGVIQGLNDSIIDLEELSIIKTYIKWPWYVWLAIGFAIIIFILILGWVFFMTGCCGCCCGCFGIIPLMSKCGKKSSYYTTFDNDVVT